(19) United States
(12) Patent Application Publication
(10) Pub. No.: US 2011/0274652 A1
(43) Pub. Date: Nov. 10, 2011

(54) Methods for Treating Hepatitis C Virus Infection

(75) Inventors: Joshua Henshaw, Boston, MA (US); Varun Garg, Framingham, MA (US); Alaa M. Ahmad, Pennington, MA (US); Neeraj Dubey, Quincy, MA (US)

(73) Assignee: VERTEX PHARMACEUTICALS INCORPORATED, Cambridge, MA (US)

(21) Appl. No.: 13/100,571

(22) Filed: May 4, 2011

Related U.S. Application Data
(63) Continuation of application No. PCT/US2009/063208, filed on Nov. 4, 2009.
(60) Provisional application No. 61/111,417, filed on Nov. 5, 2008.

(51) Int. Cl. A61K 38/21; A61P 31/14

(52) U.S. Cl. 424/85.4

(57) ABSTRACT

A method of treating a patient infected with HCV comprises administering to the patient VX-950 and interferon. In the method, an interferon trough level and/or a VX-950 trough level of the patient independently are monitored. The method further includes determining whether or not to adjust: subsequent administration of VX-950 to obtain a minimum target VX-950 trough level of about 1,500 ng/mL; subsequent administration of interferon to obtain a minimum target interferon trough level of about 5,000 pg/mL; or subsequent administration of VX-950 and interferon to obtain said minimum target trough levels.

### RELATED APPLICATIONS

This Application claims priority to U.S. Provisional Application Ser. No. 61/111,417 filed on Nov. 5, 2008, the contents of which are incorporated herein by reference in their entirety.

### BACKGROUND OF THE INVENTION

Infection by Hepatitis C virus (“HCV”) is a compelling human medical problem. HCV is recognized as the causative agent for most cases of non-A, non-B hepatitis, with an estimated human sero-prevalence of 3% globally [A. Alberti et al., “Natural History of Hepatitis C,” J. Hepatology, 31, (Suppl. 1), pp. 17-24 (1999)]. Nearly four million individuals may be infected in the United States alone [M. J. Alter et al., “The Epidemiology of Viral Hepatitis in the United States, Gastroenterol. Clin. North Am., 23, pp. 437-455 (1994); M. J. Alter “Hepatitis C Virus Infection in the United States,” J. Hepatology, 31, (Suppl. 1), pp. 88-91 (1999)].

Of persons who become infected with HCV, 20-25% may be able to clear the virus after the acute infection, but 75-80% will develop chronic Hepatitis C infection. (See, e.g., preface, Frontiers in Viral Hepatitis, Ed. R F Schinazi, J-P Sommadossi, and C M Rice. p. xi. Elsevier (2003)). This usually results in recurrent and progressively worsening liver inflammation, which often leads to more severe disease states such as cirrhosis and hepatocellular carcinoma (M. C. Kew, “Hepatitis C and Hepatocellular Carcinoma”, FEMS Microbiology Reviews, 14, pp. 211-220 (1994); I. Saito et al., “Hepatitis C Virus Infection is Associated with the Development of Hepatocellular Carcinoma,” Proc. Natl. Acad. Sci. USA, 87, pp. 6547-6549 (1990)). Unfortunately, there are no broadly effective treatments for the debilitating progression of chronic HCV.

The HCV genome encodes a polyprotein of 3010-3033 amino acids (Q. L. Choo, et. al., “Genetic Organization and Diversity of the Hepatitis C Virus.” Proc. Natl. Acad. Sci. USA, 88, pp. 2451-2455 (1991); N. Kato et al., “Molecular Cloning of the Human Hepatitis C Virus Genome From Japanese Patients with Non-A, Non-B Hepatitis,” Proc. Natl. Acad. Sci. USA, 87, pp. 9524-9528 (1990); A. Takamizawa et. al., “Structure and Organization of the Hepatitis C Virus Genome Isolated From Human Carriers,” J. Virol., 65, pp. 1105-1113 (1991)]. The HCV nonstructural (NS) proteins are presumed to provide the essential catalytic machinery for viral replication. The NS proteins are derived by proteolytic cleavage of the polyprotein (R. Bartenschlager et. al., “Nonstructural Protein 3 of the Hepatitis C Virus Encodes a Serine-Type Proteinase Required for Cleavage at the NS3/4 and NS4/5 Junctions,” J. Virol., 67, pp. 3835-3844 (1993); A. Grakoui et. al., “Characterization of the Hepatitis C Virus-Encoded Serine Proteinase: Determination of Proteinase-Dependent Polyprotein Cleavage Sites,” J. Virol., 67, pp. 2832-2843 (1993); A. Grakoui et. al., “Expression and Identification of Hepatitis C Virus Polyprotein Cleavage Products,” J. Virol., 67, pp. 1385-1395 (1993); L. Tomei et. al., “NS3 is a serine protease required for processing of hepatitis C virus polyprotein”, J. Virol., 67, pp. 4017-4026 (1993)).

The HCV NS protein 3 (NS3) contains a serine protease activity that helps process the majority of the viral enzymes, and is thus considered essential for viral replication and infectivity. It is known that mutations in the yellow fever virus NS3 protease decreases viral infectivity (Chambers, T. J. et. al., “Evidence that the N-terminal Domain of Nonstructural Protein NS3 From Yellow Fever Virus is a Serine Protease Responsible for Site-Specific Cleavages in the Viral Polyprotein”, Proc. Natl. Acad. Sci. USA, 87, pp. 8898-8902 (1990)). The first 181 amino acids of NS3 (residues 1027-1207 of the viral polyprotein) have been shown to contain the serine protease domain of NS3 that processes all four downstream sites of the HCV polyprotein (C. Lin et al., “Hepatitis C Virus NS3 Serine Proteinase: Trans-Cleavage Requirements and Processing Kinetics”, J. Virol., 68, pp. 8147-8157 (1994)).

The HCV NS3 serine protease and its associated cofactor, NS4A, help process all of the viral enzymes, and is thus considered essential for viral replication. This processing appears to be analogous to that carried out by the human immunodeficiency virus aspartyl protease, which is also involved in viral enzyme processing. HIV protease inhibitors, which inhibit viral protein processing, are potent antiviral agents in man, indicating that interrupting this stage of the viral life cycle results in therapeutically active agents. Consequently it is an attractive target for drug discovery.

There are not currently any satisfactory anti-HCV agents or treatments. Until recently, the only established therapy for HCV disease was interferon treatment. The first approved therapy for HCV infection was treatment with standard (non-pegylated) interferon alfa. However, interferons have significant side effects (M. A. Wlaker et al., “Hepatitis C Virus: An Overview of Current Approaches and Progress,” DDT, 4, pp. 518-29 (1999); D. Moradpour et al., “Current and Evolving Therapies for Hepatitis C,” Eur. J. Gastroenterol. Hepatol., 11, pp. 1199-1202 (1999); H. L. A. Janssen et al. “Suicide Associated with Alfa-Interferon Therapy for Chronic Viral Hepatitis,” J. Hepatol., 21, pp. 241-243 (1994); P. F. Renault et al., “Side Effects of Alpha Interferon,” Seminars in Liver Disease, 9, pp. 273-277. (1989)) and interferon alfa monotherapy induces long term remission in only a fraction (˜25%) of cases (O. Weiland, “Interferon Therapy in Chronic Hepatitis C Virus Infection”, FEMS Microbiol. Rev., 14, pp. 279-288 (1994)). The addition of ribavirin to the treatment regimen increases response rates slightly. Recent introductions of the pegylated forms of interferon (PEG-INTRON® and PEGASYS®), which has also been combined with ribavirin have resulted in only modest improvements in remission rates and only partial reductions in side effects. The current standard of care is a treatment regimen lasting 24-48 weeks, depending on prognostic factors such as HCV genotype and demonstration of initial response to therapy. Moreover, the prospects for effective anti-HCV vaccines remain uncertain.

Thus, there is a need for new methods of treating HCV infection, in particular which can prevent or minimize patient's HCV viral breakthrough.

### SUMMARY OF THE INVENTION

The present invention provides methods for treating HCV infections.

In one embodiment, the present invention is directed to a method of treating a patient infected with HCV. The method comprises administering to the patient VX-950, and administering to the patient interferon. In the method, an interferon trough level and/or a VX-950 trough level of the patient independently are monitored. The method further includes determining whether or not to adjust: subsequent administration of VX-950 to obtain a minimum target VX-950 trough level of about 1,500 ng/mL; subsequent administration of interferon to obtain a minimum target interferon trough level of about 5,000 pg/mL; or subsequent administration of VX-950 and interferon to obtain said minimum target trough levels.

In another embodiment, the present invention is directed to a method of treating a population of patients who are infected with HCV, wherein less than about 25% population of the patients undergo HCV viral breakthrough. The method comprises administering to the patients VX-950, and administering to the patient interferon. In the method, an interferon trough level and/or a VX-950 trough level of the patient independently are monitored. The method further includes determining whether or not to adjust: subsequent administration of VX-950 to obtain a minimum target VX-950 trough level of about 1,500 ng/mL; subsequent administration of interferon to obtain a minimum target interferon trough level of about 5,000 pg/mL; or subsequent administration of VX-950 and interferon to obtain said minimum target trough levels. When at least one of the monitored interferon and VX-950 trough levels does not meet its target level, subsequent administration of VX-950 and/or of interferon in each patient independently is adjusted to meet the target level.

The present invention also provides use of VX-950 and interferon for treating a patient infected with HCV through monitoring independently an interferon trough level and/or a VX-950 trough level of the patient; and through determining whether or not to adjust: subsequent administration of VX-950 to obtain a minimum target VX-950 trough level of about 1,500 ng/mL; subsequent administration of interferon to obtain a minimum target interferon trough level of about 5,000 pg/mL; or subsequent administration of VX-950 and interferon to obtain said minimum target trough levels.

The present invention also provides use of VX-950 and interferon for the manufacture of a medicament for treating a patient infected with HCV through monitoring independently an interferon trough level and/or a VX-950 trough level of the patient; and through determining whether or not to adjust: subsequent administration of VX-950 to obtain a minimum target VX-950 trough level of about 1,500 ng/mL; subsequent administration of interferon to obtain a minimum target interferon trough level of about 5,000 pg/mL; or subsequent administration of VX-950 and interferon to obtain said minimum target trough levels.

With Applicants' invention, treatment for an HCV infection can be done with less than about 25% (e.g., less than about 10% or less than about 7%, less than about 5%, or less than about 3%) HCV viral breakthrough.

### DESCRIPTION OF THE DRAWINGS

The FIGURE is a scatter plot showing relationship between genotype 1 HCV viral breakthrough and VX-950 and Peg-IFN-alfa-2a exposure in one embodiment of the invention.

### DETAILED DESCRIPTION OF THE INVENTION

This invention relates to methods of treating HCV infection with VX-950 (telaprevir, TVR) in combination with interferon (IFN) in a patient, such as a treatment naïve patient. Without being bound to a particular theory, Applicants have discovered that HCV infection can be treated with less than about 25% (e.g., less than about 10% or less than about 7%, less than about 5%, or less than about 3%) HCV viral breakthrough, when an interferon trough level of at least about 5,000 pg/mL, or a VX-950 trough level of at least about 1,500 ng/mL, or both is achieved during treatment.

As used herein, the term “trough level” refers to the concentration of a drug in plasma just before the next dose, or the minimum drug concentration between two doses. As used herein, the term “viral breakthrough” is defined as plasma HCV RNA increase by 1−log₁₀ after an initial decline, or HCV RNA equal to, or greater than, 100 IU/ml after becoming undetectable, whilst the patient is still undergoing drug treatment. As used herein, HCV RNA being “undetectable” means that the HCV RNA is present in less than 10 IU/ml as determined by assays currently commercially available, and preferably as determined by the Roche COBAS TaqMan™ HCV/HPS assay.

VX-950 is described in PCT Publication Numbers WO 02/018369 and WO 2006/050250, and PCT Serial Number PCT/US2008/006572, filed on May 21, 2008, with reference to the following structural formula, or a pharmaceutically acceptable salt thereof:

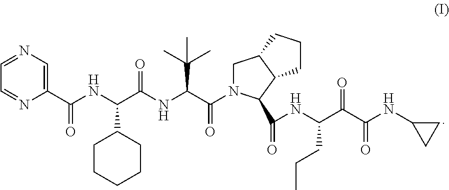

Other descriptions of VX-950 can be found in PCT Publication Numbers WO 07/098,270 and WO 08/106,151.

Suitable examples of interferon that can be employed in the invention include Albuferon™ (albumin-Interferon alpha) available from Human Genome Sciences; PEG-INTRON® (peginterferon alfa-2b, available from Schering Corporation, Kenilworth, N.J.); INTRON-A®, (VIRAFERON®, interferon alfa-2b available from Schering Corporation, Kenilworth, N.J.); PEGASYS® (peginterferon alfa-2a available Hoffmann-La Roche, Nutley, N.J.); ROFERON® (recombinant interferon alfa-2a available from Hoffmann-La Roche, Nutley, N.J.); BEREFOR® (interferon alfa 2 available from Boehringer Ingelheim Pharmaceutical, Inc., Ridgefield, Conn.); SUMIFERON® (a purified blend of natural alpha interferons such as Sumiferon available from Sumitomo, Japan); WELLFERON® (interferon alpha n1 available from Glaxo Wellcome Ltd., Great Britain); ALFERON® (a mixture of natural alpha interferons made by Interferon Sciences, and available from Purdue Frederick Co., CT); alpha-interferon; natural alpha interferon 2a; natural alpha interferon 2b; pegylated alpha interferon 2a or 2b; consensus alpha interferon (Amgen, Inc., Newbury Park, Calif.); REBETRON® (Schering Plough, Interferon-alpha 2B+Ribavirin); pegylated interferon alpha (Reddy, K. R. et al. “Efficacy and Safety of Pegylated (40-kd) Interferon alpha-2a Compared with Interferon alpha-2a in Noncirrhotic Patients with Chronic Hepatitis C, Hepatology, 33, pp. 433-438 (2001)); consensus interferon (INFERGEN®) (Kao, J. H., et al., “Efficacy of Consensus Interferon in the Treatment of Chronic Hepatitis,” J. Gastroenterol. Hepatol. 15, pp. 1418-1423 (2000); lymphoblastoid or “natural” interferon; interferon tau (Clayette, P. et al., “IFN-tau, A New Interferon Type I with Antiretroviral activity,” Pathol. Biol. (Paris) 47, pp. 553-559 (1999)); and Omega Duros® delivering omega interferon via implantable Duros® (Intarcia Therapeutics, Inc., Mountain View, Calif.).

In one embodiment, the present invention is directed to a method of treating a patient infected with HCV by administering VX-950 and administering interferon, to the patient. During treatment, an interferon trough level and/or a VX-950 trough level of the patient is monitored independently. Depending upon the observed trough level(s) of VX-950 and/or interferon, a determination is made whether or not to adjust: subsequent administration of VX-950 to obtain a minimum target VX-950 trough level of about 1,500 ng/mL; subsequent administration of interferon to obtain a minimum target interferon trough level of about 5,000 pg/mL; or subsequent administration of VX-950 and interferon to obtain said minimum target trough levels. In a specific embodiment, when at least one of the monitored interferon and VX-950 trough levels does not meet its target level, subsequent administration of VX-950 and/or interferon is adjusted to meet the target level.

In another embodiment, the present invention is directed to a method of treating a population of patients who are infected with HCV, wherein less than about 25% population of the patients undergo HCV viral breakthrough. VX-950 and interferon are administered to each of the patients independently. During treatment, an interferon trough level and/or a VX-950 trough level of the patients is monitored independently. Depending upon the observed trough levels of VX-950 and interferon, for each patient, an independent determination is made whether or not to adjust: subsequent administration of VX-950 to obtain a minimum target VX-950 trough level of about 1,500 ng/mL; subsequent administration of interferon to obtain a minimum target interferon trough level of about 5,000 pg/mL; or subsequent administration of VX-950 and interferon to obtain said minimum target trough levels. When at least one of the monitored interferon and VX-950 trough levels does not meet its target level, subsequent administration of VX-950 and/or interferon in each patient independently is adjusted to meet the target level.

In a specific embodiment of the methods of the invention, an interferon trough level is monitored during treatment. Depending upon the observed trough level of interferon, a determination is made whether or not to adjust subsequent administration of interferon to obtain a minimum target interferon trough level of about 5,000 pg/mL. In a further specific embodiment, when the monitored interferon trough level does not meet its target level, subsequent administration of interferon is adjusted to meet the target level.

In another specific embodiment of the methods of the invention, a VX-950 trough level is monitored during treatment. Depending upon the observed trough level of VX-950, a determination is made whether or not to adjust subsequent administration of VX-950 to obtain a minimum target VX-950 trough level of about 1,500 ng/mL. In a further specific embodiment, when the monitored VX-950 trough level does not meet its target level, subsequent administration of VX-950 is adjusted to meet the target level.

In yet another specific embodiment of the methods of the invention, a VX-950 trough level and an interferon trough level are independently monitored during treatment. Depending upon the observed trough levels of VX-950 and interferon, an independent determination is made whether or not to adjust: subsequent administration of VX-950 to obtain a minimum target VX-950 trough level of about 1,500 ng/mL; subsequent administration of interferon to obtain a minimum target interferon trough level of about 5,000 pg/mL; or subsequent administration of VX-950 and interferon to obtain said minimum target trough levels. In a further specific embodiment, when at least one of the monitored interferon and VX-950 trough levels does not meet its target level, subsequent administration of VX-950 and/or interferon in each patient independently is adjusted to meet the target level.

Typically, the target interferon trough level is at least about 5,000 pg/mL. In a specific embodiment, the target interferon trough level is at least about 7,000 pg/mL. In another specific embodiment, the target interferon trough level is at least about 10,000 pg/mL. In yet another specific embodiment, the target interferon trough level is in a range of between about 5,000 pg/mL and about 40,000 pg/mL. In yet another specific embodiment, the target interferon trough level is in a range of between about 7,000 pg/mL and about 40,000 pg/mL. In yet another specific embodiment, the target interferon trough level is in a range of between about 10,000 pg/mL and about 40,000 pg/mL.

Typically, the target VX-950 trough level is at least about 1,500 ng/mL. In a specific embodiment, the target VX-950 trough level is at least about 1,700 ng/mL. In another specific embodiment, the target VX-950 trough level is at least about 2,000 ng/mL. In yet another specific embodiment, the target VX-950 trough level is at least about 2,200 ng/mL. In yet another specific embodiment, the target VX-950 trough level is in a range of between about 1,500 ng/mL and about 5,000 ng/mL. In yet another specific embodiment, the target VX-950 trough level is in a range of between about 1,700 ng/mL and about 5,000 ng/mL. In yet another specific embodiment, the target VX-950 trough level is in a range of between about 2,000 ng/mL and about 5,000 ng/mL. In yet another specific embodiment, the target VX-950 trough level is in a range of between about 2,200 ng/mL and about 5,000 ng/mL.

In yet another specific embodiment, the target interferon trough level is at least about 5,000 pg/mL, at least about 7,000 pg/mL or at least about 10,000 pg/mL; and the target VX-950 trough level is at least about 1,700 ng/mL. In yet another specific embodiment, the target interferon trough level is at least about 5,000 pg/mL, at least about 7,000 pg/mL or at least about 10,000 pg/mL; and the target VX-950 trough level is at least about 2,000 ng/mL. In yet another specific embodiment, the target interferon trough level is at least about 5,000 pg/mL, at least about 7,000 pg/mL or at least about 10,000 pg/mL; and the target VX-950 trough level is at least about 2,200 ng/mL.

In yet another specific embodiment, the target interferon trough level is in a range of between about 5,000 pg/mL and about 40,000 pg/mL; and the target VX-950 trough level is in a range of between about 1,500 ng/mL and about 5,000 ng/mL, such as between about 1,700 ng/mL and about 5,000 ng/mL, between about 2,000 ng/mL and about 5,000 ng/mL, or between about 2,200 ng/mL and about 5,000 ng/mL. In yet another specific embodiment, the target interferon trough level is in a range of between about 7,000 pg/mL and about 40,000 pg/mL; and the target VX-950 trough level is in a range of between about 1,500 ng/mL and about 5,000 ng/mL, such as between about 1,700 ng/mL and about 5,000 ng/mL, between about 2,000 ng/mL and about 5,000 ng/mL, or between about 2,200 ng/mL and about 5,000 ng/mL. In yet another specific embodiment, the target interferon trough level is in a range of between about 10,000 pg/mL and about 40,000 pg/mL; and the target VX-950 trough level is in a range of between about 1,500 ng/mL and about 5,000 ng/mL, such as between about 1,700 ng/mL and about 5,000 ng/mL, between about 2,000 ng/mL and about 5,000 ng/mL, or between about 2,200 ng/mL and about 5,000 ng/mL.

It is noted that, in the invention, when at least one of the target interferon and VX-950 trough levels is achieved, the other trough level is not required to achieve its target trough level. In a first example, if a patient's interferon trough level of greater than, or equal to, the target interferon level is achieved, the patient's VX-950 trough level can be lower than the target VX-950 trough level. In the first example, for instance, the patient's VX-950 trough level can be at least about 500 ng/mL (e.g., at least about 750 ng/mL, a level of between about 500 ng/mL and about 1,500 ng/mL, a level of between about 750 ng/mL and about 1,500 ng/mL, a level of between about 1,000 ng/mL and about 1,500 ng/mL). In a second example, if a patient's VX-950 trough level of greater than, or equal to, the target VX-950 level is achieved, the patient's interferon trough level can be lower than the target interferon trough level. In the second example, for instance, the patient's interferon trough level can be at least about 1,000 pg/mL (e.g., at least about 2,500 pg/mL, a level of between about 1,000 pg/mL and about 5,000 pg/mL, a level of between about 2,500 pg/mL and about 5,000 pg/mL, a level of between about 3,000 pg/mL and about 5,000 pg/mL). Alternatively, a certain embodiment of the invention provides a method in which both of the target interferon and VX-950 trough levels are achieved.

Generally in the invention, VX-950 and interferon are administered independently. For example, VX-950 and interferon can be administered separately or together. Typically, at least one dosage form comprising VX-950 is administered to a patient over a 24-hour period so as to achieve and/or maintain the VX-950 target trough level over the 24-hour period. Typically, at least one dosage form comprising interferon is administered to a patient weekly so as to achieve and/or maintain the target interferon trough level over the week period. However, it is noted that, as desired, the VX-950 dosage form(s) can be administered every other day, or every three days, etc., so as to achieve and/or maintain the target VX-950 trough level. Similarly, it also is noted that, as desired, the interferon dosage form(s) can be administered to a patient every 5 days, or every four days, etc., so as to achieve and/or maintain the target interferon trough level. In a specific embodiment, the achieved target VX-950 and interferon trough levels independently are maintained over at least about 25% (e.g., at least about 50% or at least about 75%) of their respective treatment period (e.g., VX-950 treatment period or interferon treatment period). In yet another specific embodiment, the achieved target VX-950 and interferon trough levels independently are maintained over their respective entire treatment period. Generally, the time period during which the target trough level is maintained is a period of at least about 4 weeks, such as at least about 8 weeks, at least about 12 weeks or at least about 24 weeks. When the achieved target trough level (either VX-950 or interferon) is maintained over a portion of the respective treatment period, preferably, the achieved target trough level is maintained at an initial phase of the respective treatment period.

In some embodiments, VX-950 and interferon are administered over the entire treatment period. In these embodiments, the VX-950 treatment period and the interferon treatment period are the same.

Alternatively, in some embodiments, VX-950 and interferon are administered over two phases, an initial phase and a secondary phase. VX-950 may be administered in either the initial or secondary phase, or in both phases. In some embodiments, VX-950 is administered only in the initial phase, and interferon is administered in both of the initial and secondary phases. Alternatively, in some other embodiments, VX-950 is administered only in the secondary phase, and interferon is administered in both of the initial and secondary phases.

Suitable, specific examples of duration of the initial and secondary phases can be found in PCT/US 2008/006572, filed on May 21, 2005. For instance the initial phase can be a period of at least about 4 weeks, or between about 4 weeks and about 24 weeks (e.g., about 4 weeks, about 6 weeks, about 8 weeks, about 10 weeks, about 12 weeks, about 16 weeks, about 20 weeks, about 24 weeks, etc.), and the secondary phase can be at least about 12 weeks, e.g., the secondary phase can be about 12 weeks to about 36 weeks. In certain embodiments, the secondary phase is about 12 weeks. In other embodiments, the secondary phase is about 24 weeks. In still other embodiments, the secondary phase is about 36 weeks. In certain embodiments, the sum of the initial and secondary phase is about 24 weeks to about 48 weeks (such as about 24, 36, or 48 weeks). In some embodiments, the initial and secondary phases can be identical in duration.

In some embodiments, a method of this invention comprises administering VX-950 and interferon independently for a period of between about 4 weeks and about 12 weeks (e.g., about 4, 6, 8 or 12 weeks), for a period of between about 20 weeks and about 28 weeks (e.g., about 20, 24, or 28 weeks), or for a period of between about 8 weeks and about 24 weeks (e.g., about 8, 12, 16 or 24 weeks). In one aspect of each of these embodiments, the administration of VX-950 and interferon independently (initial phase) is followed by administration of interferon without VX-950 (secondary phase) for a period of between about 4 weeks and about 36 weeks (e.g., between about 8 weeks and about 36 weeks, between about 8 weeks and about 24 weeks, between about 4 weeks and about 24 weeks). Specific exemplary regimens include: administering VX-950 and interferon independently for about 8 weeks followed by administering interferon without VX-950 for about 16 weeks for a total treatment regimen of about 24 weeks; administering VX-950 and interferon independently for about 12 weeks followed by administering interferon without VX-950 for about 12 weeks for a total treatment regimen of about 24 weeks. In such regimens, optionally is provided administration of ribavirin for all (for both initial and secondary phases), or a part of each regimen (e.g., only for the initial phase or only for the secondary phase).

In certain embodiments, VX-950 and interferon are administered independently for less than about 12 weeks.

In certain embodiments, VX-950 and interferon are administered independently for about 8-12 weeks.

In certain embodiments, VX-950 and interferon are administered independently for about 10 weeks.

In certain embodiments, VX-950 and interferon are administered independently for less than about 10 weeks.

In certain embodiments, VX-950 and interferon are administered independently for about 2 weeks.

In other embodiments, VX-950 and interferon are administered independently for less than about 8 weeks (or about 8 weeks), less than about 6 weeks (or about 6 weeks), or less than about 4 weeks (or about 4 weeks).

In one embodiment, a method of this invention comprises administering VX-950, interferon and ribavirin independently for about 12 weeks.

In one embodiment, a method of this invention comprises administering VX-950, interferon and ribavirin independently for about 12 weeks (initial phase), followed by administering interferon and ribavirin independently for about 12 weeks (secondary phase).

In one embodiment, a method of this invention comprises administering VX-950, interferon and ribavirin independently for about 12 weeks (initial phase), followed by administering interferon and ribavirin independently for about 36 weeks (secondary phase).

In one embodiment, a method of this invention comprises administering a combination of VX-950, interferon and ribavirin independently for about 24 weeks (initial phase), followed by administering interferon and ribavirin independently for about 24 weeks (secondary phase).

In one embodiment, a method of the invention comprises administering VX-950 and interferon independently for about two weeks (initial phase), followed by about 22 weeks of administration of interferon (secondary phase).

In one embodiment, a method of the invention comprises administration of VX-950 and interferon independently for about two weeks (initial phase), followed by about 46 weeks of administration of interferon and ribavirin independently (secondary phase).

In one embodiment, a method of the invention comprises administration of VX-950 and interferon independently for about two weeks (initial phase), followed by about 22 weeks of administration of interferon and ribavirin independently (secondary phase).

In one embodiment, a method of the invention comprises the administration of VX-950 and interferon independently for about two weeks (initial phase), followed by about 46 weeks of administration of interferon and ribavirin independently (secondary phase).

In one embodiment, a method of the invention comprises the administration of VX-950, interferon and ribavirin independently for about two weeks (initial phase), followed by about 22 weeks of administration of interferon and ribavirin independently (secondary phase).

In one embodiment, a method of the invention comprises administration of VX-950, interferon and ribavirin independently for about two weeks (initial phase), followed by about 46 weeks of administration of interferon and ribavirin independently (secondary phase).

In one embodiment, a method of the invention comprises administration of VX-950 and interferon independently for about four weeks (initial phase), followed by about 20 weeks of administration of interferon and ribavirin independently (secondary phase).

In one embodiment, a method of the invention comprises administration of VX-950 and interferon independently for about four weeks (initial phase), followed by about 44 weeks of administration of interferon and ribavirin independently (secondary phase).

In one embodiment, a method of the invention comprises administration of VX-950, interferon and ribavirin independently for about four weeks (initial phase), followed by about 20 weeks of administration of interferon and ribavirin independently (secondary phase).

In one embodiment, a method of the invention comprises administration of VX-950, interferon and ribavirin independently for about four weeks (initial phase), followed by about 44 weeks of administration of interferon and ribavirin (secondary phase).

In some embodiments, any of the initial phases described above can be conducted for about 12 weeks and the secondary phases can be conducted for about 12 weeks. Alternatively, the initial phase can be conducted for about 12 weeks and the secondary phase can be conducted for about 24 weeks. In still other aspects, the initial phase can be conducted for about 12 weeks, and the secondary phase can be conducted for about 36 weeks.

In some embodiments, any of the initial phases described above can be conducted for about 8 weeks and the secondary phases can be conducted for about 16 weeks. Alternatively, the initial phase can be conducted for about 8 weeks and the secondary phase can be conducted for about 40 weeks. In still other aspects, the initial phase can be conducted for about 8 weeks and the secondary phase can be conducted for about 40 weeks.

In some embodiments, any of the initial and secondary phases described above can be switched with each other, for example, to administer interferon (optionally with ribavirin) in the initial phase, and administer VX-950 and interferon independently (optionally with ribavirin) in the secondary phase.

In some embodiments, the method includes administering VX-950 and interferon independently for less than about 48 weeks. For instance, the method includes administering VX-950 and interferon independently for less than about 24 weeks. For instance, the method includes administering VX-950 and interferon independently for less than about 12 weeks.

In some embodiments, the method includes administering VX-950 and interferon and ribavirin independently for less than about 48 weeks. For instance, the method includes administering VX-950, interferon and ribavirin independently for less than about 24 weeks. For instance, the method includes administering VX-950, interferon and ribavirin independently for less than about 12 weeks.

Generally, various dosage forms, formulation types and administration frequencies, and combinations thereof can be employed in the invention to adjust VX-950 and interferon trough levels. Any suitable dosage form and formulation type can be employed in the invention.

Various types of interferon, including various dosage forms and formulation types, that can be employed in the invention are commercially available (see, e.g., specific examples of interferon described above). For example, various types of interferon are commercially available in vials as a prepared, premeasured solution or as a lyophilized (freeze-dried) powder with a separate diluent (mixing fluid). Pegylated interferon alfa-2b (Peg-Intron®) and alfa-2a (Pegasys®) vary from the other interferons by having molecules of polyethylene glycol (PEG) attached to them. The PEG is believed to cause the interferon to remain in the body longer and thus prolongs the effects of the interferon as well as its effectiveness. Pegylated interferon is generally administered by injection under the skin (subcutaneous). Pegasys® comes as an injectable solution in pre-filled syringes or in vials. The usual dose of Pegasys® is 180 μg, taken once a week. PEG-Intron® generally comes in a pre-filled pen that contains powder and sterile water; pushing down on the pen mixes them together. The dose of PEG-Intron® generally depends on weight-1.5 μg per kilogram (a range of between about 50 and about 150 total), taken once a week. In certain embodiments, a pegylated interferon, e.g., pegylated interferon-alpha 2a or pegylated interfero-alpha 2b, is employed in the invention. Typically, interferon can be dosed according to the dosage regimens described in its commercial product labels.

Specific examples of dosage forms and formulation types of VX-950 that can be employed in the invention include those described in WO 2005/123076, WO 2007/109604, WO 2007/109605, WO 2008/080167, WO 2006/050250 and PCT/US 2008/006572, filed on May 21, 2008. For example, the amount of VX-950 in its dosage forms can be from about 100 mg to about 1500 mg, from about 300 mg to about 1500 mg, from about 300 mg to about 1250 mg, about 450 mg, about 750 mg, or about 1250 mg. Each of these dosage forms can be administered, e.g., once, twice, or three times per day, as desired. Each of these dosage forms can be in one or more dosage forms (e.g., ampule, capsule, cream, emulsion, fluid, grain, drop, injection, suspension, tablet, powder). Each of these dosage forms can be administered by one or more routes (e.g., orally, by infusion, by injection, topically, or parenterally) as considered appropriate by a skilled person in the art and depending on the dosage form.

Generally, in the invention, the overall amounts of VX-950 per administration according to the present invention can be administered in a single dosage form or in more than one dosage form. Similarly, the overall amounts of interferon per administration according to the present invention can be administered in a single dosage form or in more than one dosage form. If in separate dosage forms, each dosage form is administered simultaneously or in any time period around administration of the other dosage forms. Separate dosage forms may be administered in any order. That is, any dosage forms may be administered prior to, together with, or following the other dosage forms. For instance, for dosing regimens calling for dosing more than once a day, one or more pill or dose may be given at each time per day (e.g., 1 pill, three times per day or 3 pills, three times per day). In some embodiments of this invention will employ at least 2 pills per dose).

In some embodiments, a specified dosage form of VX-950 (e.g., a dosage form comprising VX-950 in an amount of from about 300 mg to about 1500 mg) is administered, e.g., once a day, twice a day (e.g., BID; q12h), 3 times a day (e.g., TID; q8h), or 4 times a day.

In a certain embodiment, a dosage form comprising 450 mg of VX-950 is employed in the invention. The 450 mg form of VX-950 can be administered, for example, 3 times per day (e.g., every 8 hours) or 4 times per day (e.g., every 6 hours).

In a certain embodiment, a dosage form comprising 750 mg of VX-950 is employed in the invention. The 750 mg form of VX-950 can be administered, for example, 3 times per day (e.g., every 8 hours) or 4 times per day (e.g., every 6 hours).

In a certain embodiment, a dosage form comprising 1250 mg of VX-950 is employed in the invention. The 1250 mg form of VX-950 can be administered, for example, 2 times per day (e.g., every 12 hours) or 3 times per day (e.g., every 8 hours).

In a certain embodiment, VX-950 is in a solid dosage form, such as tablet or powder form. In another certain embodiment, VX-950 is in a tablet form (e.g., about 250 mg tablet).

In the invention, VX-950 and an interferon can be administered separately or together. Generally, VX-950 may be administered orally, parenterally, sublingually, by inhalation spray, topically, rectally, nasally, buccally, vaginally or via an implanted reservoir. Interferon is not typically administered orally, although orally administered forms are in development. Nevertheless, nothing herein limits the methods or combinations of this invention to any specific dosage forms or regime. As recognized by skilled practitioners, dosages of interferon are typically measured in IU (e.g., about 4 million IU to about 12 million IU). Interferon may also be dosed by micrograms. For example, a standard dose of Peg-Intron is about 1.0-1.5 μg/kg/wk and of Pegasys is about 180 μg/wk.

In a certain embodiment, VX-950 is administered orally or intravenously. In another certain embodiment, VX-950 is administered orally.

Optionally one or more other therapeutic agents, i.e., compounds having a therapeutic utility, can be administered to a patient in combination with VX-950 and interferon, or both. The additional therapeutic agent(s) can be administered simultaneously or separately, as part of a regimen of VX-950, interferon, or both.

In some embodiments, the additional therapeutic agent (other than VX-950 and interferon) is selected from an immunomodulatory agent; an antiviral agent; an inhibitor of HCV protease (other than VX-950); an inhibitor of another target in the HCV life cycle (other than NS3/4A protease); an inhibitor of internal ribosome entry, a broad-spectrum viral inhibitor; or a cytochrome P-450 inhibitor; or combinations thereof. The additional therapeutic agent can also be an inhibitor of viral cellular entry.

Specific examples of the additional therapeutic agents that can be employed in the invention include anti-HCV agents (other than VX-950), such as ribavirin, amantadine, and telbivudine; other inhibitors of hepatitis C proteases (NS2-NS3 inhibitors and NS3-NS4A inhibitors); inhibitors of other targets in the HCV life cycle, including helicase, polymerase, and metalloprotease inhibitors; inhibitors of internal ribosome entry; broad-spectrum viral inhibitors, such as IMPDH inhibitors (e.g., compounds described in U.S. Pat. Nos. 5,807,876, 6,498,178, 6,344,465, and 6,054,472; and PCT publications WO 97/40028, WO 98/40381, and WO 00/56331; and mycophenolic acid and derivatives thereof, and including, but not limited to, VX-497, VX-148, and VX-944); or any of their combinations.

In some embodiments, the additional therapeutic agent is ribavirin. Certain specific examples of ribavirin treatment regimens in combination with VX-950 and interferon are described above. Ribavirin is typically administered orally, and tablet forms of ribavirin are currently commercially available. General standard, daily dose of ribavirin tablets (e.g., about 200 mg tablets) is about 800 mg to about 1200 mg. Nevertheless, nothing herein limits the methods or combinations of this invention to any specific dosage forms or regime. Typically, ribavirin can be dosed according to the dosage regimens described in its commercial product labels.

Other agents (e.g., non-immunomodulatory or immunomodulatory compounds) may be used in the invention include, but are not limited to, those specified in WO 02/18369 (see, e.g., page 273, lines 9-22 and page 274, line 4 to page 276, line 11).

Still other agents include those described in various published U.S. patent applications. These publications provide additional teachings of compounds and methods that could be used in combination with VX-950 in the methods of this invention, particularly for the treatment of hepatitis. It is contemplated that any such methods and compositions may be used in combination with the methods and compositions of the present invention. For brevity, the disclosure the disclosures from those publications is referred to be reference to the publication number but it should be noted that the disclosure of the compounds in particular is specifically incorporated herein by reference. Examples of such publications include U.S. patent application Publication Nos.: US 20040058982, US 20050192212, US 20050080005, US 20050062522, US 20050020503, US 20040229818, US 20040229817, US 20040224900, US 20040186125, US 20040171626, US 20040110747, US 20040072788, US 20040067901, US 20030191067, US 20030187018, US 20030186895, US 20030181363, US 20020147160, US 20040082574, US 20050192212, US 20050187192, US 20050187165, US 20050049220, and US 20050222236.

A cytochrome P450 monooxygenase (“CYP”) inhibitor can inhibit metabolism of VX-950. Therefore, the cytochrome P450 monooxygenase inhibitor would be in an amount effective to inhibit metabolism of VX-950. Accordingly, the CYP inhibitor is administered in an amount such that the bioavailability of or exposure to VX-950 is increased in comparison to VX-950 in the absence of the CYP inhibitor. CYP inhibitors include, but are not limited to, ritonavir (WO 94/14436), ketoconazole, troleandomycin, 4-methylpyrazole, cyclosporin, clomethiazole, cimetidine, itraconazole, fluconazole, miconazole, fluvoxamine, fluoxetine, nefazodone, sertraline, indinavir, nelfinavir, amprenavir, fosamprenavir, saquinavir, lopinavir, delavirdine, erythromycin, VX-944, and VX-497. Preferred CYP inhibitors include ritonavir, ketoconazole, troleandomycin, 4-methylpyrazole, cyclosporin, and clomethiazole.

Methods for measuring the ability of a compound to inhibit cytochrome P50 monooxygenase activity are known (see, U.S. Pat. No. 6,037,157, and Yun et al., Drug Metabolism & Disposition, 21, 403-407 (1993)). Methods for evaluating the influence of co-administration of VX-950 and a CYP inhibitor in a subject are also known (US 2004/0028755). Any such methods could be used in connection with this invention to determine the pharmacokinetic impact of a combination.

For the CYP inhibitors, the dosage levels of between about 0.001 to about 200 mg/kg body weight per day, would be typical. More typical would be dosage levels of between about 0.1 to about 50 mg/kg or about 1.1 to about 25 mg/kg per day.

For specific dosage forms of ritonavir, see U.S. Pat. No. 6,037,157, and the documents cited therein: U.S. Pat. No. 5,484,801, U.S. patent application Ser. No. 08/402,690, and PCT Publications Nos. WO 95/07696 and WO 95/09614.

Generally in the invention, “administration” or “co-administration” of one or more therapeutic agents (including VX-950, interferon and ribavirin, and any combination thereof) includes administering each active therapeutic agent in the same dosage form or in different dosage forms. When administered in different dosage forms, the active therapeutic agent may be administered at different times, including simultaneously or in any time period around administration of the other dosage forms. Separate dosage forms may be administered in any order. That is, any dosage forms may be administered prior to, together with, or following the other dosage forms.

VX-950 and any additional agent, may be formulated in separate dosage forms. Alternatively, to decrease the number of dosage forms administered to a patient, VX-950 and any additional agent, may be formulated together in any combination. Any separate dosage forms may be administered at the same time or different times. It should be understood that dosage forms should be administered within a time period such that the biological effects were advantageous.

If pharmaceutically acceptable salts are employed in the invention as active therapeutic agents, those salts are typically derived from inorganic or organic acids and bases. Included among such acid salts are the following: acetate, adipate, alginate, aspartate, benzoate, benzene sulfonate, bisulfate, butyrate, citrate, camphorate, camphor sulfonate, cyclopentane-propionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium and magnesium salts, salts with organic bases, such as dicyclohexylamine salts, N-methyl-D-glucamine, and salts with amino acids such as arginine, lysine, and so forth.

Also, the basic nitrogen-containing groups may be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained.

In the invention, as desired, modification of therapeutic agent(s) can also be employed by, for example, appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

Typically, one or more therapeutic agents, including VX-950 and interferon, employed in the invention are included in pharmaceutical compositions, though the therapeutic agent(s) may be administered alone. A “pharmaceutical composition” means a composition comprising a therapeutic agent disclosed herein, and at least one component selected from the group comprising pharmaceutically acceptable carriers, diluents, coatings, adjuvants, excipients, or vehicles, such as preserving agents, fillers, disintegrating agents, wetting agents, emulsifying agents, emulsion stabilizing agents, suspending agents, isotonic agents, sweetening agents, flavoring agents, perfuming agents, coloring agents, antibacterial agents, antifungal agents, other therapeutic agents, lubricating agents, adsorption delaying or promoting agents, and dispensing agents, depending on the nature of the mode of administration and dosage forms. The compositions may be presented in the form of tablets, pills, granules, powders, aqueous solutions or suspensions, injectable solutions, elixirs or syrups.

Exemplary suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Exemplary antibacterial and antifungal agents for the prevention of the action of microorganisms include parabens, chlorobutanol, phenol, sorbic acid, and the like. Exemplary isotonic agents include sugars, sodium chloride and the like. Exemplary adsorption delaying agents to prolong absorption include aluminum monosterate and gelatin. Exemplary adsorption promoting agents to enhance absorption include dimethyl sulphoxide and related analogs. Exemplary carriers, diluents, solvents, vehicles, solubilizing agents, emulsifiers and emulsion stabilizers, include water, chloroform, sucrose, ethanol, isopropyl alcohol, ethyl carbonate, ethyl acetate, benzyl alcohol, tetrahydrofurfuryl alcohol, benzyl benzoate, polyols, propylene glycol, 1,3-butylene glycol, glycerol, polyethylene glycols, dimethylformamide, Tween 60, Span&commat; 80, cetostearyl alcohol, myristyl alcohol, glyceryl mono-stearate and sodium lauryl sulfate, fatty acid esters of sorbitan, vegetable oils (such as cottonseed oil, groundnut oil, corn germ oil, olive oil, castor oil and sesame oil) and injectable organic esters such as ethyl oleate, and the like, or suitable mixtures of these substances. Exemplary excipients include lactose, milk sugar, sodium citrate, calcium carbonate, dicalcium phosphate phosphate. Exemplary disintegrating agents include starch, alginic acids and certain complex silicates. Exemplary lubricants include magnesium stearate, sodium lauryl sulphate, talc, as well as high molecular weight polyethylene glycols.

The choice of material in the pharmaceutical composition other than the therapeutic agent is generally determined in accordance with the chemical properties of the therapeutic agent, such as solubility, the particular mode of administration and the provisions to be observed in pharmaceutical practice. For example, excipients such as lactose, sodium citrate, calcium carbonate, dicalcium phosphate and disintegrating agents such as starch, alginic acids and certain complex silicates combined with lubricants such as magnesium stearate, sodium lauryl sulphate and talc may be used for preparing tablets.

The pharmaceutical compositions may be presented in assorted forms such as tablets, pills, granules, powders, aqueous solutions or suspensions, injectable solutions, elixirs or syrups.

“Liquid dosage form” means the dose of the therapeutic agent to be administered to the patient is in liquid form, for example, pharmaceutically acceptable emulsions, solutions, suspensions, syrups and elixirs. In addition to the active compounds, the liquid dosage forms may contain inert diluents commonly used in the art, such solvents, solubilizing agents and emulsifiers.

Solid compositions may also be employed as fillers in soft and hard-filled gelatin capsules using such excipients as lactose or milk sugar as well as high molecular weight polyethylene glycols, and the like.

When aqueous suspensions are used they can contain emulsifying agents or agents which facilitate suspension.

The oily phase of the emulsion pharmaceutical composition may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier that acts as a stabilizer. It is also preferred to include both an oil and a fat. Together, the emulsifier (s) with or without stabilizer (s) make up the emulsifying wax, and the way together with the oil and fat make up the emulsifying ointment base which forms the oily dispersed phase of the cream formulations.

If desired, the aqueous phase of the cream base may include, for example, a least 30% w/w of a polyhydric alcohol, i.e. an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound that enhances absorption or penetration of the active ingredient through the skin or other affected areas.

The choice of suitable oils or fats for a formulation is based on achieving the desired cosmetic properties. Thus the cream should preferably be a non-greasy, non-staining and washable product with suitable consistency to avoid leakage from tubes or other containers.

Straight or branched chain, mono- or di-basic alkyl esters such as di-isopropyl myristate, decyl oleate, isopropyl palmitate, butyl stearate, 2-ethylhexyl palmitate or a blend of branched chain esters known as Crodamol CAP may be used. These may be used alone or in combination depending on the properties required. Alternatively, high melting point lipids such as white soft paraffin and/or liquid paraffin or other mineral oils can be used.

Generally, a therapeutic agent/pharmaceutical compositions disclosed herein may be administered in a suitable formulation to humans and animals by topical or systemic administration, including oral, inhalational, rectal, nasal, buccal, sublingual, vaginal, colonic, parenteral (including subcutaneous, intramuscular, intravenous, intradermal, intrathecal and epidural), intracisternal and intraperitoneal. It will be appreciated that the preferred route may vary with for example the condition of the recipient.

“Pharmaceutically acceptable dosage forms” refers to dosage forms of a therapeutic agent (including VX-950) disclosed herein, and includes, for example, tablets, powders, elixirs, syrups, liquid preparations, including suspensions, sprays, inhalants tablets, lozenges, emulsions, solutions, granules, capsules and suppositories, as well as liquid preparations for injections, including liposome preparations. Techniques and formulations generally may be found in Remington's Pharmaceutical Sciences, Mack Publishing Co., Easton, Pa., latest edition.

“Formulations suitable for oral administration” may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tables may be prepared by compressing in a suitable machine the active ingredient in a free-flowing form such as a powder or granules, optionally mixed with a binder, lubricant, inert diluent, preservative, surface active or dispersing agent. Moulded tablets may be made by moulding in a suitable machine a mixture of the powdered compounds moistened with an inert liquid diluent. The tablets may optionally be coated or scored and may be formulated so as to provide slow or controlled release of the active ingredient therein.

Solid compositions for rectal administration include suppositories formulated in accordance with known methods and containing at least one compound of the invention.

If desired, and for more effective distribution, a therapeutic agent disclosed herein can be microencapsulated in, or attached to, a slow release or targeted delivery systems such as a biocompatible, biodegradable polymer matrices (e.g., poly (d, l-lactide co-glycolide)), liposomes, and microspheres and subcutaneously or intramuscularly injected by a technique called subcutaneous or intramuscular depot to provide continuous slow release of the compound (s) for a period of 2 weeks or longer. The therapeutic agent may be sterilized, for example, by filtration through a bacteria retaining filter, or by incorporating sterilizing agents in the form of sterile solid compositions which can be dissolved in sterile water, or some other sterile injectable medium immediately before use.

“Formulations suitable for nasal or inhalational administration” means formulations which are in a form suitable to be administered nasally or by inhalation to a patient. The formulation may contain a carrier, in a powder form, having a particle size for example in the range 1 to 500 microns (including particle sizes in a range between 20 and 500 microns in increments of 5 microns such as 30 microns, 35 microns, etc.) Suitable formulations wherein the carrier is a liquid, for administration as for example a nasal spray or as nasal drops, include aqueous or oily solutions of the active ingredient. Formulations suitable for aerosol administration may be prepared according to conventional methods and may be delivered with other therapeutic agents. Inhalational therapy is readily administered by metered dose inhalers.

“Formulations suitable for oral administration” means formulations which are in a form suitable to be administered orally to a patient. The formulations may be presented as discrete units such as capsules, cachets or tablets each containing a predetermined amount of the active ingredient; as a powder or granules; as solution or a suspension in an aqueous liquid or a non-aqueous liquid; or as an oil-in-water liquid emulsion or a water-in-oil liquid emulsion. The therapeutic agent may also be presented as a bolus, electuary or paste.

“Formulations suitable for parenteral administration” means formulations that are in a form suitable to be administered parenterally to a patient. The formulations are sterile and include emulsions, suspensions, aqueous and non-aqueous injection solutions, which may contain suspending agents and thickening agents and anti-oxidants, buffers, bacteriostats and solutes which render the formulation isotonic, and have a suitably adjusted pH, with the blood of the intended recipient.

“Formulations suitable for rectal or vaginal administrations” means formulations that are in a form suitable to be administered rectally or vaginally to a patient. The formulation is preferably in the form of suppositories that can be prepared by mixing the compounds of this invention with suitable non-irritating excipients or carriers such as cocoa butter, polyethylene glycol or a suppository wax, which are solid at ordinary temperatures but liquid at body temperature and therefore, melt in the rectum or vaginal cavity and release the active component.

“Formulations suitable for systemic administration” means formulations that are in a form suitable to be administered systemically to a patient. The formulation is preferably administered by injection, including transmuscular, intravenous, intraperitoneal, and subcutaneous. For injection, the compounds of the invention are formulated in liquid solutions, preferably in physiologically compatible buffers such as Hank's solution or Ringer's solution. In addition, the compounds may be formulated in solid form and redissolved or suspended immediately prior to use. Lyophilized forms are also included. Systematic administration also can be by transmucosal or transdermal means, or the compounds can be administered orally. For transmucosal or transdermal administration, penetrants appropriate to the barrier to be permeated are used in the formulation. Such penetrants are generally known in the art, and include, for example, bile salts and fusidic acid derivatives for transmucosal administration. In addition, detergents may be used to facilitate permeation. Transmucosal administration may be through use of nasal sprays, for example, or suppositories. For oral administration, the compounds are formulated into conventional oral administration forms such as capsules, tablets, and tonics.

“Formulations suitable for topical administration” means formulations that are in a form suitable to be administered topically to a patient. The formulation may be presented as a topical ointment, salves, powders, sprays and inhalants, gels (water or alcohol based), creams, as is generally known in the art, or incorporated into a matrix base for application in a patch, which would allow a controlled release of compound through the transdermal barrier. When formulated in an ointment, the active ingredients may be employed with either a paraffinic or a water-miscible ointment base. Alternatively, the active ingredients may be formulated in a cream with an oil-in-water cream base. Formulations suitable for topical administration in the eye include eye drops wherein the active ingredient is dissolved or suspended in a suitable carrier, especially an aqueous solvent for the active ingredient. Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

“Solid dosage form” means the dosage form of a therapeutic agent disclosed herein is solid form, for example capsules, tablets, pills, powders, dragees or granules. In such solid dosage forms, the compound of the invention is admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alignates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example paraffin, (f) absorption accelerators, as for example, quaternary ammonium compounds, (g) wetting agents, as for example, cetyl alcohol and glycerol monostearate, (h) adsorbents, as for example, kaolin and bentonite, (i) lubricants, as for example, talc, calcium stearate, magnesium stearate, solid polyethylene glycols, sodium lauryl (j) opacifying agents, (k) buffering agents, and agents which release the compound (s) of the invention in a certain part of the intestinal tract in a delayed manner.

The amount of active therapeutic agent(s) that may be combined with the carrier and/or excipient materials to produce a single dosage form will vary depending upon the host treated and the particular mode of administration. A typical preparation will contain from about 5% to about 95% active therapeutic agent (w/w). Preferably, such preparations contain from about 20% to about 80% therapeutic agent.

The formulations can be prepared in unit dosage form by any of the methods well known in the art of pharmacy. Such methods include the step of bringing into association the active ingredient with the carrier that constitutes one or more accessory ingredients. In general the formulations are prepared by uniformly and intimately bringing into association the active ingredient with liquid carriers or finely divided solid carriers or both, and then, if necessary, shaping the product.

The formulations may be presented in unit-dose or multi-dose containers, for example sealed ampoules and vials with elastomeric stoppers, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

The pharmaceutical compositions and dosage formulations disclosed herein are preferably for use in vivo. Nevertheless, this is not intended as a limitation to using of the pharmaceutical compositions and dosage formulations for any purpose. For example, a biological substance pre-treated with a pharmaceutical composition disclosed herein can also be employed in the invention. Such biological substances include, but are not limited to, blood and components thereof such as plasma, platelets, subpopulations of blood cells and the like; organs such as kidney, liver, heart, lung, etc; sperm and ova; bone marrow and components thereof, and other fluids to be infused into a patient such as saline, dextrose, etc.

It should also be understood that a specific dosage and treatment regimen for any particular patient will depend upon a variety of factors, including the activity of the specific compound employed, the age, body weight, general health, sex, diet, time of administration, rate of excretion, drug combination, the judgment of the treating physician and the severity of the particular disease being treated, prior treatment history, co-morbidities or concomitant medications, baseline viral load, race, duration of diseases, status of liver function and degree of liver fibrosis/cirrhosis, and the goal of therapy (eliminating circulating virus per-transplant or viral eradication). The amount of active ingredients will also depend upon the particular described compound and the presence or absence and the nature of the additional anti-viral agent in the composition.

According to the treatment regimens and dosage forms of this invention, co-therapy of VX-950 and interferon is effective to decrease the viral load in a sample or in a patient, wherein said virus encodes a NS3/4A serine protease necessary for the viral life cycle (or in an amount effective to carry out a method of this invention). Accordingly, the invention also provides a method for treating a patient infected with a virus characterized by a virally encoded NS3/4A serine protease that is necessary for the life cycle of the virus by administering to said patient VX-950 and interferon (and optionally one or more additional therapeutic agent) as described above.

In the invention, each active therapeutic agent employed in the invention, independently, can be administered to a patient with or without food. In some embodiments, VX-950 and/or interferon independently are administered with food. Without being bound to a particular theory, administration of VX-950 and/or interferon in combination with food may enhance its absorption in the gastrointestinal tract and increase its bioavailability. As used herein, the phrase “in combination with food” means that VX-950 and/or interferon independently are administered within about 90 minutes of consumption of food, e.g., no more than about 90 minutes after food is eaten and no more than about 90 minutes prior to eating period. In some embodiments, VX-950 and/or interferon independently are administered up to about 30 minutes before, or up to 30 minutes after consumption of food. Although not required, and any type of food (high fat or low fat) can be consumed, a high-fat meal may provide improved absorption as compared to lower fat meals. As used herein, “high fat” means food in which over about 30% of the calories are provided by fat. In a certain embodiment, the food has at least about 50 calories. In another certain embodiment, the food has at least about 100 calories. In yet another certain embodiment, the food has at least about 50-100 calories up to about 3,000 calories, up to about 2,000 calories, or up to about 1,000 calories. In yet another certain embodiment, the food includes at least about 30% of its total calories from fat.

Generally in the invention, treatment may completely eradicate the HCV viral infection or reduce the severity thereof. In some embodiments, a method of this invention involves achieving, relatively rapidly, a therapeutically effective plasma concentration of VX-950 and then maintaining the trough level such that an effective therapeutic response is achieved. An effective therapeutic response may be, for example, one or both of a) achieving a sustained viral response; and b) achieving undetectable HCV RNA in the plasma by at least about 12 weeks (about 12 weeks or more). The term “undetectable” is as defined above.

In other embodiments, a method of this invention treats a patient infected with HCV such that the level of HCV RNA in the patient after the administration is at least about 2 log₁₀ (e.g., at least about 4 log₁₀) lower than that before treatment.

A relatively rapid drop in viral plasma concentration may be obtained by administering a loading dose to a patient. In one embodiment, the loading dose is about 1250 mg of VX-950.

In some embodiments, the method of this invention is able to achieve week 4 RVR and week 12 undetectable status.

Generally in the invention, a “patient” includes a mammal, particularly a human being.

In certain embodiments, a method of the invention provides treatment of a patient infected with genotype 1 Hepatitis C virus. It is generally believed that genotype 1 HCV infection is the most difficult strain of HCV to treat and the most prevalent strain in the United States.

Advantageously, both HCV treatment naïve and previously treated patients benefit from the methods of this invention. For the avoidance of doubt, patients that may be treated according to the methods of this invention include those where HCV treatment has not been tried or has failed, including non-responding, rebound, relapse, and breakthrough patients. In certain embodiments, the methods of the present invention treat HCV treatment naïve patient. As used herein, an “HCV treatment naïve” patient means that the patient has no previous HCV treatment with a drug(s) approved, or seeking approval, by the U.S. Food and Drug Administration (FDA), or any other U.S. or international agency equivalent to the U.S. FDA.

The methods of the invention can be used as a chronic or acute therapy. As would be realized by skilled practitioners, if a method of this invention is being used to treat a patient prophylactically, and that patient becomes infected with Hepatitis C virus, the method may then treat the infection. Therefore, one embodiment of this invention provides methods for treating or preventing a Hepatitis C infection in a patient.

The assay for determined VX-950 and interferon concentrations in patient's plasma can be performed by methods well known in the art. See, e.g., Wasley, A. et al., Semin. Liver Dis., 20:1-16, 2000; Alter, H. J. et al., Semin. Liver Dis., 20: 17-35, 2000; Brown, R. S. Jr. et al., Liver Transpl., 9: S10-S13, 2003; DeFrancesco, R. et al., Nature, 436(7053): 953-960, 2005; Bowen, D. G. et al., J. Hepatol., 42: 408-417, 2005; Hoofnagle, J. H., Hepatology, 36: S21-S29, 2002, Brown, R. S. Jr. et al., Nature, 436 (7053): 973-978, 2005; and Chisari, F. V., Nature, 436(7053): 930-932, 2005.

In order that this invention is more fully understood, the following preparative and testing examples are set forth. These examples are for the purpose of illustration only and are not to be construed as limiting the scope of the invention in any way.

### EXEMPLIFICATION

### Formulations of VX-950

VX-950 may be prepared in general by methods known to those skilled in the art (see, e.g., WO 02/18369). Details of the preparation of the formulations of Examples 1-7 are as described in WO 2005/123075. Details of the preparation of the formulations of Examples 8-19 are as described in WO 2007/109604. Details of the preparation of the formulations of Examples 20-23 are as described in WO 2007/109605. Details of the preparation of the formulations of Example 24 are as described in WO 2008/080167. For example, various amounts of HPMCAS-HG (Hypromellose Acetate Succinate, HG grade, Shin-Etsu Chemical Co.) polymer, HPMC-60SHSO (Metolose, Shin-Etsu Chemical Co.) polymer and SLS (Sodium Lauryl Sulfate, Sigma/Fisher) surfactant were used; and spray drying and subsequent post-drying in a qualified vacuum dryer were performed.

### Example 1

A solid dispersion was prepared comprising the following ingredients (percentage of total weight):

| | |
|---|---|
| VX-950 | 49.5% |
| HPMC 40 cp | 49.5% |
| SLS | 1% |

The composition 1 was prepared by dissolving VX-950, HPMC, and SLS in methanol:methylene chloride (1:1) followed by evaporation of the solvents using rotation evaporation under vacuum. The product was milled to particles with mean particle size of about 200 μm.

### Example 2

A solid dispersion was prepared comprising the following ingredients (percentage of total weight):

| | |
|---|---|
| VX-950 | 49.5% |
| HPC | 49.5% |
| SLS | 1% |

The composition 2 was prepared by dissolving VX-950 and HPC in methylene chloride. SLS was suspended in the solution. The solvent was then evaporated by rotation evaporation under vacuum. The product was milled to particles with mean particle size of about 200 μm.

### Example 3

A solid dispersion was prepared comprising the following ingredients (percentage of total weight):

| | |
|---|---|
| VX-950 | 49.5% |
| PVP K30 | 49.5% |
| SLS | 1% |

The composition 3 was prepared by dissolving VX-950, PVP K30, and suspending SLS in methanol:methylene chloride followed by spray-drying to remove the solvent. The mean particle size of the product is about 150 μm.

### Example 4

A solid dispersion was prepared comprising the following ingredients (percentage of total weight):

| | |
|---|---|
| VX-950 | 49.5% |
| HPMCP | 49.5% |
| SLS | 1% |

The composition 4 was prepared by using a similar procedure as in example 3. The mean particle size of the product is about 150 μm.

### Example 5

An oral dosage formulation was prepared as follows. VX-950 and PVP K29/32 were dissolved in methylene chloride, then sodium lauryl sulfate was added and dispersed in the solution to form a homogenous suspension. This suspension was spray-dried using an inlet temperature of 90° C. and an outlet temperature of 56° C., and the product was collected from the cyclone. The spray-dried dispersion was fluid-bed dried at 75° C. for 8 hours.

VX-950 Solid Dispersion

| % (w/w) | Ingredient | |
|---|---|---|
| 49.5 | VX-950 | Spray-dried |
| 49.5 | PVP K29/32 | from a MeCl₂ |
| 1 | SLS | solution |

The solid dispersion was suspended in a 1% HPMC, 0.002% simethicone solution using a steel rotary mixer. The resultant suspension is physically and chemically stable at the concentrations of 0.8-50 mg/ml VX-950 for at least 24 hours. The powder is then suspended and dosed within 24 hrs as described in the table below.

Suspension Vehicle

| % | Ingredient | Function |
|---|---|---|
| 1 | Low viscosity hydroxypropyl | Suspending agent |
| | methylcellulose | |
| 0.002 | Simethicone | Anti-foam |
| 99 | Water | diluent |

### Example 6

Dispersions in single dose glass vials mixed with 1% HPMC vehicle were dosed. The solid residue remaining in the vial was 0.8%-4% compared to 28%-56% when dosed in a syringe mixed with water. Dispersions dosed were: VX950/PVPK-30/SLS (tox. lot, refreshed), VX950/HPMCAS/SLS/SDBS (spray dried at ISP starting with crystalline DS containing 5% PVPK-30), VX950/HPMC E15/10% Vit E TPGS, VX950/PVP-VA/10% Vit E TPGS. The results of these studies are provided below.

| Formulation ID | Mean Cmax | Mean Tmax | |
|---|---|---|---|
| (30 mg/Kg dose) | (ng/mL) | (hr) | Mean % F |
| 1:1 VX950:PVPK30, | 981 ± 200 | 0.6 ± 0.3 | 19.6 ± 3.1 |
| 1% SLS (Refreshed Tox.) | | | |
| Niro-49% HPMCAS/1% | 980 ± 200 | 0.9 ± 0.3 | 29.5 ± 4.8 |
| SLS/1% SDBS/49% VX-950 | | | |
| 40.5% PVP-VA/10% | 1482 ± 400 | 0.5 ± 0.0 | 29.8 ± 9.1 |
| ETPGS/49.5% VX-950 | | | |
| 40.5% HPMC/10% | 1890 ± 400 | 0.4 ± 0.1 | 34.7 ± 7.8 |
| ETPGS/49.5% VX-950 | | | |

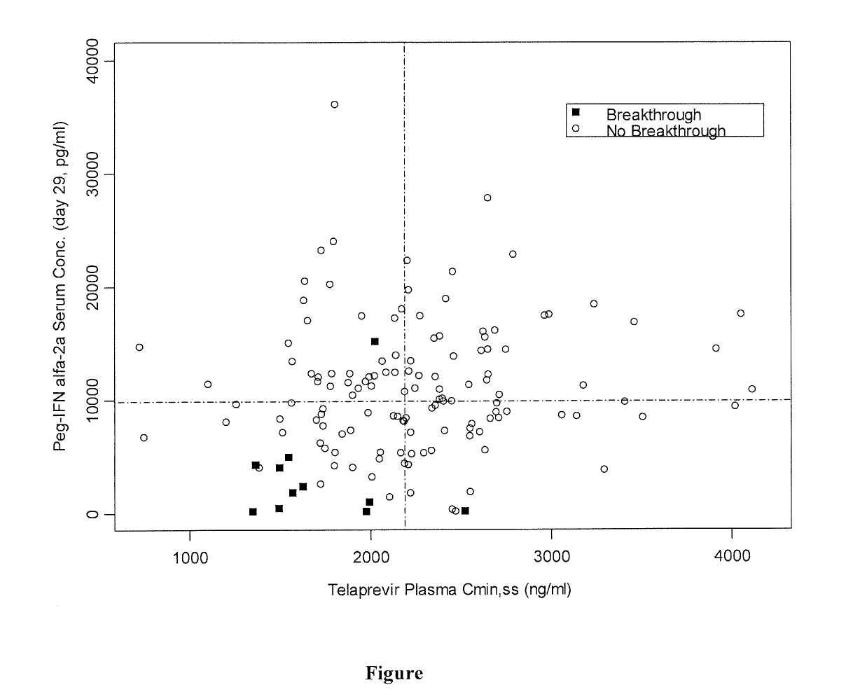

As can be seen in the above table, HPMC E-15/10% Vit ETPGS had the highest Cmax and % F (FIG. 2). PVP-VA/10% Vit ETPGS had the second highest Cmax and % F. HPMCAS exhibited a somewhat sustained release profile with a Cmax comparable to PVPK-30 refreshed dispersion and a % F comparable to PVP-VA.

### Example 7

Three formulations were manufactured on the SD Micro spray drier (100 gm). The first 2 formulations had the same ingredients, but varied in acetone levels. The third formulation was a polymer mixture of HPC and HPMC phthalate (2:1). All three formulations contained 1% SLS and 1% SDBS and drug substance that had 5% PVPK-30.

Dissolution of the polymers required homogenization, and all 3 formulations spray-dried very easily. All formulations had detectable residual solvents after manufacture, but both solvents were easily removed with oven drying (60° C.). The addition of acetone appeared to have lowered the initial content of methylene chloride. Residual solvents levels are summarized below

Residual solvents from dispersions
manufacture at ISP (100 gm scale)

| | | | Dry- | Residual | |
|---|---|---|---|---|---|
| | | | ing | Methylene | Residual |
| Lot | Formu- | solvent | Time | Chloride | Acetone |
| # | lation | Ratio | (hr) | (ppm) | (ppm) |
| 2702- | 49% VX950, | 100% | 0 | 10064 | <100 ppm |
| 801 | 49% HPMCAS, | methylene | 1 | 114 | <100 ppm |
| | 1% SLS, | Chloride | 2 | <100 ppm | <100 ppm |
| | 1% SDBS | | 63 | <100 ppm | <100 ppm |
| 2702- | 49% VX950, | 30% Acetone/ | 0 | 2889 | 1869 |
| 802 | 49% HPMCAS, | 70% methylene | 1 | <100 ppm | <100 ppm |
| | 1% SLS, | chloride | 2 | <100 ppm | <100 ppm |
| | 1% SDBS | | 63 | <100 ppm | <100 ppm |
| 2702- | 49% VX950, | 30% Acetone/ | 0 | 5641 | <100 ppm |
| 803 | 16% HPPh, | 70% methylene | 1 | <100 ppm | <100 ppm |
| | 33% HPC, | chloride | 2 | <100 ppm | <100 ppm |
| | 1% SLS, | | 63 | <100 ppm | <100 ppm |
| | 1% SDBS | | | | |

### Example 8

A solid dispersion was prepared comprising the following ingredients:

TABLE 1A
Formulation composition: 55/24.4/19.6/1 w/w/w/w VX-
950/HPMCAS-HG/HPMC-60SH/SLS) per 1.250 kg of VX-950
(22.727 kg total batch size; 2.273 kg total solids).

| Dispersion | | |
|---|---|---|
| Component | | |
| Function | Dispersion Component | kg |
| API | VX-950 | 1.250 |
| Polymer/ | Hydroxypropyl Methylcellulose Acetate | 0.555 |
| Dispersant I | Succinate, JPE (Biddle Sawyer or Shin-Etsu | |
| | HPMCAS-HG grade) | |
| Polymer/ | Hydroxypropyl Methylcellulose 60SH 50 cP | 0.445 |
| Dispersant II | (Biddle Sawyer or Shin-Etsu Metolose, | |
| | HPMC60SH50) | |
| Surfactant | Sodium Lauryl Sulfate (SLS) | 0.023 |
| Process Solvent | Methylene Chloride, NF | 16.363 |
| | (for Dispersion) | |
| Process Solvent | Acetone, NF | 4.091 |
| | (for Dispersion) | |

### Example 9

A solid dispersion was prepared comprising the following ingredients:

TABLE 2A
Formulation composition: 55/14.7/29.3/1 w/w/w/w VX-
950/HPMCAS-HG/HPMC-60SH/SLS) per 1.250 kg of VX-950
(22.727 kg total batch size; 2.273 kg total solids).

| Dispersion | | |
|---|---|---|
| Component | | |
| Function | Dispersion Component | kg |
| API | VX-950 | 1.250 |
| Polymer/ | Hydroxypropyl Methylcellulose Acetate | 0.334 |
| Dispersant I | Succinate, JPE (Biddle Sawyer or Shin-Etsu | |
| | HPMCAS-HG grade) | |
| Polymer/ | Hydroxypropyl Methylcellulose 60SH 50 cP | 0.666 |
| Dispersant II | (Biddle Sawyer or Shin-Etsu Metolose, | |
| | HPMC60SH50) | |
| Surfactant | Sodium Lauryl Sulfate (SLS) | 0.023 |
| Process Solvent | Methylene Chloride, NF | 16.363 |
| | (for Dispersion) | |
| Process Solvent | Acetone, NF | 4.091 |
| | (for Dispersion) | |

### Example 10

A solid dispersion was prepared comprising the following ingredients:

TABLE 3A
Formulation composition: 60/24.4/14.6/1 w/w/w/w VX-
950/HPMCAS-HG/HPMC-60SH/SLS per 1.250 kg of VX-950
(20.83 kg total batch size; 2.083 kg total solids).

| Dispersion | | |
|---|---|---|
| Component | | |
| Function | Dispersion Component | kg |
| API | VX-950 | 1.250 |
| Polymer/ | Hydroxypropyl Methylcellulose Acetate | 0.508 |
| Dispersant I | Succinate, JPE (Biddle Sawyer or Shin-Etsu | |
| | HPMCAS-HG grade) | |
| Polymer/ | Hydroxypropyl Methylcellulose 60SH 50 cP | 0.304 |
| Dispersant II | (Biddle Sawyer or Shin-Etsu Metolose, | |
| | HPMC60SH50) | |
| Surfactant | Sodium Lauryl Sulfate (SLS) | 0.021 |
| Process Solvent | Methylene Chloride, NF | 15.000 |
| | (for Dispersion) | |
| Process Solvent | Acetone, NF | 3.750 |
| | (for Dispersion) | |

### Example 11

A solid dispersion was prepared comprising the following ingredients:

TABLE 4A
Formulation composition: 65/17/17/1 w/w/w/w VX-950/HPMCAS-
HG/HPMC-60SH/SLS per 1.250 kg of VX-950 (19.23 kg
total batch size; 1.923 kg total solids).

| Dispersion | | |
|---|---|---|
| Component | | |
| Function | Dispersion Component | kg |
| API | VX-950 | 1.250 |
| Polymer/ | Hydroxypropyl Methylcellulose Acetate | 0.327 |
| Dispersant I | Succinate, JPE (Biddle Sawyer or Shin-Etsu | |
| | HPMCAS-HG grade) | |
| Polymer/ | Hydroxypropyl Methylcellulose 60SH 50 cP | 0.327 |
| Dispersant II | (Biddle Sawyer or Shin-Etsu Metolose, | |
| | HPMC60SH50) | |
| Surfactant | Sodium Lauryl Sulfate (SLS) | 0.019 |
| Process Solvent | Methylene Chloride, NF | 13.846 |
| | (for Dispersion) | |
| Process Solvent | Acetone, NF | 3.462 |
| | (for Dispersion) | |

### Example 12

A solid dispersion was prepared comprising the following ingredients:

TABLE 5A
Formulation composition: (70/9.7/19.3/1 w/w/w/w VX-
950/HPMCAS-HG/HPMC-60SH/SLS) per 1.250 kg of VX-950
(17.86 kg total batch size; 1.786 kg total solids).

| Dispersion | | |
|---|---|---|
| Component | | |
| Function | Dispersion Component | kg |
| API | VX-950 | 1.250 |
| Polymer/ | Hydroxypropyl Methylcellulose Acetate | 0.173 |
| Dispersant I | Succinate, JPE (Biddle Sawyer or Shin- | |
| | Etsu HPMCAS-HG grade) | |
| Polymer/ | Hydroxypropyl Methylcellulose 60SH 50 cP | 0.345 |
| Dispersant II | (Biddle Sawyer or Shin-Etsu Metolose, | |
| | HPMC60SH50) | |
| Surfactant | Sodium Lauryl Sulfate (SLS) | 0.018 |
| Process Solvent | Methylene Chloride, NF | 12.857 |
| | (for Dispersion) | |
| Process Solvent | Acetone, NF | 3.214 |
| | (for Dispersion) | |

### Example 13

A solid dispersion was prepared comprising the following ingredients:

TABLE 6A
Formulation composition: (60/39/0/1 w/w/w/w VX-950/HPMCAS-
HG/HPMC-60SH/SLS) per 1.250 kg of VX-950 (20.833
kg total batch size; 2.083 kg total solids).

| Dispersion | | |
|---|---|---|
| Component | | |
| Function | Dispersion Component | kg |
| API | VX-950 | 1.250 |
| Polymer/ | Hydroxypropyl Methylcellulose Acetate | 0.813 |
| Dispersant I | Succinate, JPE (Biddle Sawyer or Shin- | |
| | Etsu HPMCAS-HG grade) | |
| Polymer/ | Hydroxypropyl Methylcellulose 60SH 50 cP | 0.000 |
| Dispersant II | (Biddle Sawyer or Shin-Etsu Metolose, | |
| | HPMC60SH50) | |
| Surfactant | Sodium Lauryl Sulfate (SLS) | 0.021 |
| Process Solvent | Methylene Chloride, NF | 15.000 |
| | (for Dispersion) | |
| Process Solvent | Acetone, NF | 3.750 |
| | (for Dispersion) | |

### Example 14

Solid dispersions of amorphous VX-950 comprising the ingredients given below in Table 7A were prepared (as percent weight of total dispersion) and the dissolution of the solid dispersion was measured in fasted SGF at 37.5° C.

TABLE 7A
Solid Dispersions of VX-950

| Dispersion | VX-950 | HPMC AS | HPMC | SLS |
|---|---|---|---|---|
| 1 | 49.5 | 24.5 | 24.5 | 1 |
| 2 | 83 | 8 | 8 | 1 |
| 3 | 83 | 8 | 8 | 1 |
| 4 | 49.5 | 24.5 | 24.5 | 1 |

### Example 15

Solid dispersions of amorphous VX-950 comprising the ingredients given below in Table 8A were prepared (as percent weight of total dispersion) and the dissolution of the solid dispersion was measured in fasted SGF at 37.5° C.

TABLE 8A
Solid Dispersions of VX-950

| Dispersion | VX-950 | HPMC AS | HPMC | SLS |
|---|---|---|---|---|
| 1 | 70 | 14.5 | 14.5 | 1 |
| 2 | 65 | 14.6 | 19.4 | 1 |
| 3 | 65 | 9.7 | 24.3 | 1 |
| 4 | 60 | 19.5 | 19.5 | 1 |
| 5 | 60 | 14.6 | 24.4 | 1 |
| 6 | 70 | 9.7 | 19.3 | 1 |

### Example 16

Solid dispersions of amorphous VX-950 were prepared comprising the ingredients given below in Table 9A were prepared (as percent weight of total dispersion) and the dissolution of the solid dispersion was measured in fasted SGF at 37.5° C.

TABLE 9A
Solid Dispersions of VX-950

| Dispersion | VX-950 | HPMC AS | HPMC | SLS |
|---|---|---|---|---|
| 1 | 70 | 9.7 | 19.3 | 1 |
| 2 | 70 | 14.5 | 14.5 | 1 |
| 3 | 70 | 9.7 | 19.3 | 1 |
| 4 | 49.5 | 24.5 | 24.5 | 1 |
| 5 | 83 | 8 | 8 | 1 |
| 6 | 83 | 8 | 8 | 1 |
| 7 | 49.5 | 24.5 | 24.5 | 1 |

### Example 17

The following solid dispersions of amorphous VX-950 were prepared with the solvent mixtures shown in Table 10A. D50 and bulk density were determined for the dispersions. Values for content are given as percent weight.

TABLE 10A
Solid dispersions of VX-950

| ID | Formulation | Content | Solids Conc. | Process Solvent | d50 | bulk density |
|---|---|---|---|---|---|---|
| 13 | VX-950/HPMCAS-HG/SLS | 49.5/49.5/1 | 10 | 70/30 DCM/Acetone | 29.99 | 0.22 |
| 14 | VX-950/HPMCAS-HG/SLS | 49.5/49.5/1 | 10 | 70/30 DCM/Acetone | 20.13 | 0.25 |
| 15 | VX-950/HPMCAS-HG/SLS | 49.5/49.5/1 | 10 | 70/30 DCM/Acetone | 19.07 | 0.24 |
| 16 | VX-950/HPMCAS-HG/SLS | 49.5/49.5/1 | 10 | 70/30 DCM/Acetone | 31.9 | 0.24 |
| 17 | VX-950/HPMCAS-HG/SLS | 49.5/49.5/1 | 10 | 70/30 DCM/Acetone | 32.18 | 0.27 |
| 18 | VX-950/HPMCAS-HG/SLS | 49.5/49.5/1 | 10 | 70/30 DCM/Acetone | 20.67 | 0.26 |
| 19 | VX-950/HPMCAS-HG/SLS | 49.5/49.5/1 | 10 | 66.6/28.5/5 DCM/Acetone/GAA | 43.03 | 0.37 |
| 20 | VX-950/HPMCAS-HG/SLS | 49.5/49.5/1 | 10 | 63/27/10 DCM/Acetone/GAA | 47.02 | 0.41 |
| 21 | HPMCAS-HG | 100 | 6 | 70/30 DCM/Acetone | 38.33 | 0.24 |
| 22 | VX-950/HPMCAS-HG/SLS | 83/16/1 | 20 | 100 DCM | 33.58 | 0.33 |
| 23 | VX-950/HPMCAS-HG/SLS | ~82.44/15.89/1.67 | 20 | 100 DCM | 31.67 | 0.35 |
| 24 | VX-950/HPMCAS-HG/HPMC-60SH/SLS* | 49.5/24.75/24.75/1 | 10 | 77/23 DCM/Acetone | 22.98 | 0.31 |
| 25 | VX-950/HPMCAS-HG/HPMC-60SH/SLS* | 83/8/8/1 | 10 | 100 DCM | 27.72 | 0.35 |
| 26 | HPMCAS-HG | 100 | 4 | 100 DCM | 28.74 | 0.24 |
| 27 | VX-950/HPMCAS-HG/SLS | 83/16/1 | 10 | 100 DCM | 20.6 | 0.33 |
| A2341055 | VX-950 (amorphous) | 100 | 10 | 100 DCM | ? | ? |

*HPMC-60SH deemed equivalent to HPMCE50

### Example 18

A spray dried dispersion of amorphous VX-950 of the present disclosure can be used in preparing a tablet. The tablet can contain the formulation shown in Table 11A, which contains vitamin E TPGS formulated in a melt granulate:

TABLE 11A
Tableted formulation containing a
spray dried dispersion of VX-950

| | mg per | |
|---|---|---|
| Component | Tablet | Percent |
| Roller compaction blend | | |
| VX950 Spray Dried Dispersion1 | 505.1 | 74.9 |
| Pharmatose DCL 22 (Lactose, USP/NF, PhEur, [symbol] | 37.5 | 5.6 |
| Ac-Di-Sol (cross carmellose sodium, NF, PhEur, [symbol] | 24.0 | 3.6 |
| Extragranular addition | | 0.0 |
| Avicel pH 113 | 33.7 | 5.0 |
| Vitamin E TPGS (NF) | 24.0 | 3.6 |
| Ac-Di-Sol (cross carmellose sodium, NF, PhEur, [symbol] | 16.0 | 2.4 |
| Cabosil M-5 (colloidal silicon dioxide, NF, PhEur) | 8.0 | 1.2 |
| Sodium Stearyl fumarate (NF, PhEur, JP) | 26.0 | 3.9 |
| Total Formulation weight | 674.3 | 100.0 |

[symbol]

indicates data missing or illegible when filed

### Example 19

The examples in Table 12A are spray dried dispersions containing amorphous VX-950 that can be prepared: (Percents by weight are shown)

TABLE 12A
Solid Dispersions of VX-950

| VX-950 | HPMC AS | HMPC | SLS |
|---|---|---|---|
| 60 | 24.6 | 14.4 | 1 |
| 60 | 39 | 0 | 1 |
| 49.5 | 49.5 | 0 | 1 |

### Example 20

A mixture of the following components was spray dried to provide a solid dispersion of VX-950. VX-950/HPMCAS-HG/SLS was combined in a ratio of 49.5/49.5/1 wt/wt and combined in a solvent system at a solid concentration of 10, where the solvent system included methylene chloride/acetone/glacial acetic acid in a ratio of 66.6/28.5/5 to provide a product having a d50 of 43.03 and a bulk density of 0.37.

### Example 21

A mixture of the following components was spray dried to provide a solid dispersion of VX-950. VX-950/HPMCAS-HG/SLS was combined in a ratio of 49.5/49.5/1 wt/wt and combined in a solvent system at a solid concentration of 10, where the solvent system included methylene chloride/acetone/glacial acetic acid in a ratio of 63/27/10 to provide a product having a d50 of 47.02 and a bulk density of 0.41.

### Example 22

Spray dried dispersions of VX-950 were prepared using with multiple VX-950 lots, HPMCAS-HG (Hypromellose Acetate Succinate, HG grade, Shin-Etsu Chemical Co.) polymer, and SLS (Sodium Lauryl Sulfate, Fisher) surfactant. Spray drying and subsequent post-drying in a biconical dryer were performed. Dry dispersion with low residual solvent levels and target powder properties were manufactured. Success criteria included having acceptable process yield (>80%), and meeting all target drug product specifications for purity, and matching the target properties within the range specified for physical characteristics (particle size and bulk density).

Formulation Composition and Process Outline

The overall formulation composition for each of two active dispersion manufactures is described in Table 1B.

TABLE 1B
Formulation composition of each of the two active dispersion
manufactures based off of 116.25 kg VX-950 at 13 wt %.

| Component Function | Component | kg |
|---|---|---|
| API | VX-950 | 116.25 |
| Polymer/Dispersant | Hypromellose Acetate Succinate, | 116.25 |
| | NF/JPE (HPMCAS-HG) | |
| Surfactant | Sodium Lauryl Sulfate, NF (SLS) | 2.348 |
| Process Solvent | Methylene Chloride, NF | 1178.8 |
| | (for Dispersion) | |
| Process Solvent | Acetone, NF | 377.2 |
| | (for Dispersion) | |
| Process Solvent | DI Water | 15.7 |

### Example 23

### Objectives and Success Criteria

Dry dispersion with low residual solvent levels and target powder properties are manufactured. Success criteria include having acceptable process yield (>80%), and meeting all target drug product specifications for purity, and matching the target properties within the range specified for physical characteristics (particle size and bulk density).

Formulation Composition and Process Outline

The overall formulation composition for the two active dispersion manufactures is described in Table 2B.

TABLE 2B
Formulation composition of the first active dispersion
manufacture based off of 100 kg VX-950 at 15 wt %.

| Component Function | Component | kg |
|---|---|---|
| API | VX-950 | 200.0 |
| Polymer/Dispersant | Hypromellose Acetate | 100.0 |
| | Succinate, NF/JPE | |
| | (HPMCAS-HG) | |
| Surfactant | Sodium Lauryl Sulfate, | 2.02 |
| | NF (SLS) | |
| Process Solvent | Methylene Chloride, NF | 858.6 |
| | (for Dispersion) | |
| Process Solvent | Acetone, NF | 274.7 |
| | (for Dispersion) | |
| Process Solvent | DI Water | 11.4 |

### Example 24

This example provides the results of experiments in which a dispersion of VX-950 prepared by fluidized spray drying, according to that described in WO 2008/080167, was directly compressed into a tablet.

The addition of different types of Vit E and different processes for the addition of the Vit E were evaluated. The types of Vit E and methods of addition to the dispersion are shown below. A dispersion of VX-950 was prepared by fluidized spray drying as described in WO 2008/080167.

TABLE 1C
VX950 SD Tableting Experiment
Design (Potency: 250 mg VX950)

| Trial # | Vit E type | Vit E type |
|---|---|---|
| A | VitE-TPGS (24 mg) | Granulated VitE on excipients |
| C | VitE-Acetate (48 mg) | Used as is |
| E | Vit E-TPGS(24 mg) | Vit E Spray Congealed |
| F | Vit E-TPGS (24 mg) | Granulated Vit E onto VX950 |

TABLE 2C
Trial# A Formulation

| | Ingredients | Wt/Tablet | | Theoretical |
|---|---|---|---|---|
| Item | Physical mixture | (mg) | % | Qt. (g) |
| 1 | Solid Dispersion | 339.9 | 66.32 | 19.90 |
| | (73.55% VX950/26.45% HPMCAS) | | | |
| 2 | PHARMATOSE ® DCL 22 (Lactose) | 37.5 | 7.32 | 2.20 |
| 3 | AC-DI-SOL ® (Cross carmellose sodium) | 24.0 | 4.68 | 1.40 |
| 4 | Sodium Stearyl Fumarate | 1.6 | 0.32 | 0.10 |
| 5 | SLS | 3.4 | 0.66 | 0.20 |
| 6 | AVICEL ® pH 113 (Microcrystalline cellulo [symbol] | 33.7 | 6.58 | 1.97 |
| 7 | Vitamin E TPGS (granulated on excipients) | 24.0 | 4.68 | 1.40 |
| 8 | AC-DI-SOL ® (Cross carmellose sodium) | 16.0 | 3.12 | 0.94 |
| 9 | Cabosil M-5 (Colloidal silicon dioxide) | 8.0 | 1.56 | 0.47 |
| 10 | Sodium Stearyl Fumarate | 24.4 | 4.76 | 1.43 |
| | Total | 512.5 | 100 | 30.00 |

Note:
VX 950 SD Lot 02
Potency: 250 mg VX950 [symbol]
indicates data missing or illegible when filed

TABLE 3C
Trial# C Formulation

| | Ingredients | Wt/Tablet | | Theoretical |
|---|---|---|---|---|
| Item | Physical mixture | (mg) | % | Qt. (g) |
| 1 | Solid Dispersion | 339.9 | 63.36 | 79.19 |
| | (73.55% VX950/26.45% HPMCAS) | | | |
| 2 | PHARMATOSE ® DCL 22 (Lactose) | 37.5 | 6.99 | 8.74 |
| 3 | AC-DI-SOL ® (Cross carmellose sodium) | 24.0 | 4.47 | 5.59 |
| 4 | Sodium Stearyl Fumarate | 1.6 | 0.30 | 0.38 |
| 5 | SLS | 3.4 | 0.63 | 0.79 |
| 6 | AVICEL ® pH 113 (Microcrystalline cellulose) | 33.7 | 6.28 | 7.85 |
| 7 | Vitamin E-Acetate | 48.0 | 8.95 | 11.18 |
| 8 | AC-DI-SOL ® (Cross carmellose sodium) | 16.0 | 2.98 | 3.73 |
| 9 | Cabosil M-5 (Colloidal silicon dioxide) | 8.0 | 1.49 | 1.86 |
| 10 | Sodium Stearyl Fumarate | 24.4 | 4.54 | 5.68 |
| | Total | 536.5 | 100 | 125.00 |

TABLE 4C
Trial# E Formulation

| | Ingredients | Wt/Tablet | | Theoretical |
|---|---|---|---|---|
| Item | Physical mixture | (mg) | % | Qt. (g) |
| 1 | Solid Dispersion | 339.9 | 66.32 | 82.90 |
| | (73.55% VX950/26.45% HPMCAS) | | | |
| 2 | PHARMATOSE ® DCL 22 (Lactose) | 37.5 | 7.32 | 9.15 |
| 3 | AC-DI-SOL ® (Cross carmellose sodium) | 24.0 | 4.68 | 5.85 |
| 4 | Sodium Stearyl Fumarate | 1.6 | 0.32 | 0.40 |
| 5 | SLS | 3.4 | 0.66 | 0.83 |
| 6 | AVICEL ® pH 113 (Microcrystalline cellulose) | 33.7 | 6.58 | 8.22 |
| 7 | Vitamin E Spray Congealed | 24.0 | 4.68 | 5.85 |
| 8 | AC-DI-SOL ® (Cross carmellose sodium) | 16.0 | 3.12 | 3.90 |
| 9 | Cabosil M-5 (Colloidal silicon dioxide) | 8.0 | 1.56 | 1.95 |
| 10 | Sodium Stearyl Fumarate | 24.4 | 4.76 | 5.95 |
| | Total | 512.5 | 100 | 125.00 |

Note:
VX 950 SD Lot 02
Potency: 250 mg VX950

TABLE 5C
Trial# F Formulation

| | | Wt/Tablet | | Theoretical |
|---|---|---|---|---|
| Item | Ingredients | (mg) | % | Qt. (g) |
| 1 | Solid Dispersion | 339.9 | 66.32 | 66.32 |
| | (73.55% VX950/26.45% HPMCAS) | | | |
| 2 | Vitamin E granulated onto dispersion | 24.0 | 4.68 | 4.68 |
| 3 | PHARMATOSE ® DCL 22 (Lactose) | 37.5 | 7.32 | 7.32 |
| 4 | AC-DI-SOL ® (Cross carmellose sodium) | 24.0 | 4.68 | 4.68 |
| 5 | Sodium Stearyl Fumarate | 1.6 | 0.32 | 0.32 |
| 6 | SLS | 3.4 | 0.66 | 0.66 |
| 7 | AVICEL ® pH 113 (Microcrystalline cellulose) | 33.7 | 6.58 | 6.58 |
| 8 | AC-DI-SOL ® (Cross carmellose sodium) | 16.0 | 3.12 | 3.12 |
| 9 | Cabosil M-5 (Colloidal silicon dioxide) | 8.0 | 1.56 | 1.56 |
| 10 | Sodium Stearyl Fumarate | 24.4 | 4.76 | 4.76 |
| | Total | 512.5 | 100 | 100.00 |

Note:
VX 950 SD Lot 02
Potency: 250 mg VX950

TABLE 6C
VX 950 SD Lot 02 Physical parameters

| | |
|---|---|
| D10 (μm) | 13.37 |
| D50 (μm) | 51.45 |
| D90 (μm) | 141.67 |
| Bulk density (g/ml) | 0.25 |
| Tap density (g/ml) | 0.32 |

TABLE 7C
Results from the Compression Run

| | | | Tooling shape: | | | | | |
|---|---|---|---|---|---|---|---|---|
| | Tablet wt. = | | oval 0.6250 in.* | | | | | |
| | 512.5 mg | | 0.3750 in. | | | | | |
| #A-GranExcp-DC | Run #1 | Run #2 | Run #3 | Run #4 | Run #5 | Run #6 | Run #7 | Run #8 |
| Compress force (kN) | 2.37 | 2.7 | 2.84 | 4.43 | 6.95 | 8.4 | 12.5 | 16.01 |
| Eject (N) | 70 | 83 | 83 | 86 | 90 | 90 | 90 | 90 |
| Hardness (kp) | 3 | 3.2 | 3.3 | 4.8 | 6.5 | 8.5 | 10.5 | 10.8 |
| Thickness (mm) | 6.97 | 6.46 | 6.43 | 6.02 | 5.76 | 5.51 | 5.35 | 5.30 |

| | | | | Tooling shape: | | | | | |
|---|---|---|---|---|---|---|---|---|---|
| | Tablet wt. = | | | oval 0.6250 in.* | | | | | |
| | 536.5 mg | | | 0.3750 in. | | | | | |
| #C-Acet-DC | Run #1 | Run #2 | Run #3 | Run #4 | Run #5 | Run #6 | Run #7 | Run #8 | Run #9 |
| Compress force (kN) | 1.1 | 1.9 | 2.3 | 2.8 | 4.4 | 6.8 | 10.1 | 13.4 | 18.1 |
| Eject (N) | 83 | 83 | | | | | | | |
| Hardness (kp) | N/A | 2.3 | 2.5 | 2.7 | 4.4 | 6.5 | 9.9 | 13.3 | 14.5 |
| Thickness (mm) | 8.30 | 7.70 | 7.38 | 7.05 | 6.48 | 6.03 | 5.76 | 5.60 | 5.52 |

| | | | Tooling shape: | | | | |
|---|---|---|---|---|---|---|---|
| | Tablet wt. = | | oval 0.6250 in.* | | | | |
| | 512.5 mg | | 0.3750 in. | | | | |
| #E-SpCong-DC | Run #1 | Run #2 | Run #3 | Run #4 | Run #5 | Run #6 | Run #7 |
| Compress force (kN) | 1.77 | 2.23 | 3.68 | 5.61 | 8.8 | 15.5 | 18.09 |
| Eject (N) | 83 | 83 | 90 | 95 | 120 | 95 | 95.00 |
| Hardness (kp) | 2 | 2.4 | 3.6 | 6.1 | 10.4 | 14 | 14.23 |
| Thickness (mm) | 7.46 | 7.06 | 6.42 | 6.01 | 5.53 | 5.33 | 5.29 |

| | | | Tooling shape: | | | | | |
|---|---|---|---|---|---|---|---|---|
| | Tablet wt. = | | oval 0.6250 in.* | | | | | |
| #F-Gran | 512.5 mg | | 0.3750 in. | | | | | |
| VX950-DC | Run #1 | Run #2 | Run #3 | Run #4 | Run #5 | Run #6 | Run #7 | 1 |
| Compress force (kN) | 2.18 | 3.37 | 4.41 | 6.27 | 10.28 | 12.8 | 18.83 | |
| Eject (N) | 75 | 83 | 83 | 85 | 90 | 90 | 90 | |
| Hardness (kp) | 1.5 | 3.4 | 5.6 | 7.8 | 11.2 | 13.8 | 15.6 | |
| Thickness (mm) | 6.98 | 6.44 | 6.00 | 5.81 | 5.55 | 5.37 | 5.28 | |

VX-950 (Telaprevir) Combo-Therapy in Combination with Interferon and/or Ribavirin

### Example 25

### VX-950 Validation in Human Plasma

The assay for determined VX-950 concentration in human plasma can be performed by methods well known in the art. See, e.g., Wasley, A. et al., Semin. Liver Dis., 20:1-16, 2000; Alter, H. J. et al., Semin. Liver Dis., 20: 17-35, 2000; Brown, R. S. Jr. et al., Liver Transpl., 9: S10-S13, 2003; DeFrancesco, R. et al., Nature, 436(7053): 953-960, 2005; Bowen, D. G. et al., J. Hepatol., 42: 408-417, 2005; Hoofnagle, J. H., Hepatology, 36: S21-S29, 2002, Brown, R. S. Jr. et al., Nature, 436 (7053): 973-978, 2005; and Chisari, F. V., Nature, 436(7053): 930-932, 2005.

Specifically, the following VX-950 solutions were prepared and stored in capped borosilicate tubes (11.5 mL) at −20° C.:

Stock solution: 961 μg/mL of VX-950 in 2-propanol (10.0 mL)

Diluted stock solution 1: 96.1 μg/ml of VX-950 in 2-propanol (5.00 mL)

Diluted stock solution 2: 9.61 μg/ml of VX-950 in 2-propanol (10.0 mL)

Diluted stock solution 3: 0.961 μg/ml of VX-950 in 2-propanol (10.0 mL)

An internal standard stock solution was prepared to contain 1.00 mg/mL of Compound 1 (a close structural analog of VX-950) in 5.00 mL of 2-propanol, and was stored in a capped borosilicate tube (11.5 ml) at −20° C. A working solution containing the same Compound 1 was prepared to contain 300 ng/mL of Compound 1 in 100 mL of acetonitrile, and stored in a capped borosilicate bottle (100 mL) −20° C.

Sample Preparation: 100 μL of plasma and 100 μL of internal standard working solution (or acetonitrile for blank samples) were added to an extraction tube. After vortex mixing for 30 seconds, 500 μL of toluene was added and extraction was performed by vortex mixing for 30 seconds. After centrifugation at 3000 rpm at 4° C. for 5 minutes, the aqueous layer was frozen in a mixture of acetone and dry ice and the organic layer was transferred to another extraction tube. 50 μL of 2,2-dimethoxypropane was added and the samples were evaporated to dryness under nitrogen at approximately 30° C. The residue was re-dissolved in 300 μL of a mixture of heptane and acetone (90:10, v/v) [or a mixture of heptane and THF (80:20, v/v)] by vortex mixing for 60 seconds. The sample was transferred to an injection vial and an aliquot of 60 μL of the sample was injected into the chromatographic system for analysis with the following chromatographic conditions:
Mobile phase: (Isocratic elution) heptane/acetone/methanol (80:19:1, v/v/v)
Make-up solvent: acetonitrile/acetone/methanol/formic acid (40:60:1:1, v/v/v/v)
Column temperature: −1° C.
Flow rate: 1.00 mL/minute (including 0.750 mL/min mobile phase and 0.250 mL/min make-up solvent, completely transferred to detector)
Injection volume: 60 μL
Auto-sampler temperature: 3° C.

### Example 26

### Viral Breakthrough Scatter Plot

Treatment naïve subjects with genotype 1 hepatitis C received an initial 12 weeks of therapy consisting of ribavirin (COPEGUS®), dosed at 1000 or 1200 mg daily (based on weight), Peg-IFN-alfa-2a (PEGASYS®), dosed at 180 μg weekly, and telaprevir, dosed with a 1250 mg loading dose followed by a 750 mg dose every 8 hours (with 250 mg tablets). Following the first 12 weeks of therapy, subjects either received an additional 28 weeks of Peg-IFN-alfa-2a and ribavirin therapy (Group B), and additional 12 weeks of Peg-IFN-alfa-2a and ribavirin therapy (Group C), or no additional therapy (Group D). Subjects were identified who met the study breakthrough criteria, defined as an increase of >1−log₁₀ HCV RNA compared to the lowest recorded on-treatment value, or, if the HCV RNA had become undetectable, an increase to HCV RNA>100 IU/mL (confirmed by values obtained at 2 consecutive visits), monitored during the first 12 weeks of treatment (the telaprevir/placebo dosing period). Placebo subjects (Group A) were not included in this analysis.

The observed Peg-IFN-alfa-2a serum concentrations at day 29 of the study were used as representative measures of Peg-IFN-alfa-2a exposure. Telaprevir exposure was described using a population pharmacokinetic approach.

The population pharmacokinetic analysis of telaprevir was performed using nonlinear mixed effect modeling conducted with the NONMEM software (version VI. level 1.1, Globomax, Hanover, Md., USA) installed in a qualified environment. NMqual (version 6.2.0) was used to compile NONMEM runs and qualify the installation, and the compiler used was the G77 utility (Metrum institute, Augusta, Me.). The structural component of the population PK model was a one-compartment linear model. Absorption process was assumed to be governed by two functions, a Weibull-type function for the first dose followed by a 1^{st} order process for subsequent doses. The random effects were estimated on four parameters: CL/F, V/F, AK and GM. A covariance term for CL/F and V/F was included in the model. The residual variability was modeled using a combined additive and proportional error model. The covariate component of the model contained weight as a covariate on CL/F, as described by a power model. The first order conditional estimation (FOCE) with interaction method was used. Steady-state exposure measures, including the minimum telaprevir steady-state plasma concentration (C_{min,ss}), were derived from the individual Bayesian parameter estimates extracted from the model fit.

The FIGURE shows relationship between viral breakthrough and VX-950 (“Telaprevir” in the FIGURE) and Peg-IFN-alfa-2a exposure. In the figure, points represent individual subjects, open circles denote subjects who did not experience viral breakthrough, closed squares denote subjects who experienced viral breakthrough. Horizontal and vertical dashed lines represent the median values of Peg-IFN-alfa-2a and telaprevir, respectively. One subject who experienced viral breakthrough was not included in this analysis, due to this subject not having a Peg-IFN-alfa-2a concentration reported on day 29. The scatter plot of the FIGURE was produced with S-PLUS (version 7.0, Insightful, Wash., USA) for day 29 Peg-IFN-alfa-2a serum concentrations and model-predicted telaprevir C_{min,ss} for individual subjects. Subjects who experienced viral breakthrough are represented with filled squares, subjects who did not experience viral breakthrough, and were undetectable at day 85, are represented with open circles. The median day 29 Peg-IFN-alfa-2a serum concentrations and model-predicted telaprevir C_{min,ss} are represented by horizontal and vertical dashed lines, respectively.

The entire teachings of all documents (including patent and non-patent documents) cited in this application are incorporated herein by reference.

While a number of embodiments and examples of the present invention are described herein, it is apparent that these embodiments and examples may be altered to provide additional embodiments and examples which utilize the pharmaceutical formulations and drug regimens of this invention. Therefore, it will be appreciated that the scope of the present invention is to be defined by the appended claims rather than by the specific embodiments that have been represented by way of example above.

What is claimed is:

1. A method of treating a patient infected with HCV, comprising the steps of:
a) administering to the patient VX-950;
b) administering to the patient interferon;
c) monitoring an interferon trough level or a VX-950 trough level of the patient, or both; and
d) determining whether or not to adjust: i) subsequent administration of VX-950 to obtain a minimum target VX-950 trough level of about 1,500 ng/mL; ii) subsequent administration of interferon to obtain a minimum target interferon trough level of about 5,000 pg/mL; iii) or subsequent administration of VX-950 and interferon to obtain said minimum target trough levels.

2. The method of claim 1, further comprising the step of adjusting subsequent administration of VX-950 and/or of interferon when at least one of the monitored interferon and VX-950 trough levels does not meet its target level.

3. The method of claim 1, wherein the minimum target interferon trough level is about 7,000 pg/mL.

4. The method of claim 1, wherein the minimum target interferon trough level is about 10,000 pg/mL.

5. The method of claim 1, wherein the minimum target VX-950 trough level is about 1,700 ng/mL.

6. The method of claim 1, wherein the minimum target VX-950 trough level is about 2,000 ng/mL.

7. The method of claim 1, wherein the target interferon trough level is in a range of between about 5,000 pg/mL and about 40,000 pg/mL, and the target VX-950 trough level is in a range of between about 1,500 ng/mL and about 5,000 ng/mL.

8. The method of claim 7, wherein the target VX-950 trough level is in a range of between about 1,700 ng/mL and about 5,000 ng/mL.

9. The method of claim 8, wherein the target VX-950 trough level is in a range of between about 2,000 ng/mL and about 5,000 ng/mL.

10. The method of claim 1, wherein the target interferon trough level is in a range of between about 7,000 pg/mL and about 40,000 pg/mL.

11. The method of claim 10, wherein the target interferon trough level is in a range of between about 10,000 pg/mL and about 40,000 pg/mL.

12. The method of claim 1, wherein the interferon is pegylated interferon.

13. The method of claim 1, further comprising the step of administering ribavirin to the patient.

14. The method of claim 1, wherein a solid form of VX-950 is administered to the patient.

15. The method of claim 14, wherein the solid form is a tablet form.

16. The method of claim 1, wherein VX-950 is administered to the patient with food.

17. A method of treating a population of patients who are infected with HCV, comprising the steps of:
a) administering to each patient VX-950;
b) administering to the patient interferon;
c) monitoring an interferon trough level or a VX-950 trough level of the patient, or both;
d) determining whether or not to adjust: i) subsequent administration of VX-950 to obtain a minimum target VX-950 trough level of about 1,500 ng/mL; ii) subsequent administration of interferon to obtain a minimum target interferon trough level of about 5,000 pg/mL; iii) or subsequent administration of VX-950 and interferon to obtain said minimum target trough levels; and
e) adjusting independently subsequent administration of VX-950 and/or interferon in each patient when at least one of the monitored interferon and VX-950 trough levels does not meet its target level,
wherein less than about 25% of the population of the patients undergo HCV viral breakthrough.

18. The method of claim 17, wherein the minimum, target interferon trough level is about 7,000 pg/mL.

19. The method of claim 17, wherein the minimum, target interferon trough level is about 10,000 pg/mL.

20. The method of claim 17, wherein the minimum, target VX-950 trough level is about 1,700 ng/mL.

21. The method claim 17, wherein the minimum, target VX-950 trough level is about 2,000 ng/mL.

22. The method of claim 17, wherein the target interferon trough level is in a range of between about 5,000 pg/mL and about 40,000 pg/mL, and the target VX-950 trough level is in a range of between about 1,500 ng/mL and about 5,000 ng/mL.

23. The method of claim 22, wherein the target VX-950 trough level is in a range of between about 1,700 ng/mL and about 5,000 ng/mL.

24. The method of claim 23, wherein the target VX-950 trough level is in a range of between about 2,000 ng/mL and about 5,000 ng/mL.

25. The method of claim 17, wherein the target interferon trough level is in a range of between about 7,000 pg/mL and about 40,000 pg/mL.

26. The method of claim 17, wherein the target interferon trough level is in a range of between about 10,000 pg/mL and about 40,000 pg/mL.

27. The method of claim 17, wherein the interferon is pegylated interferon.

28. The method of claim 17, further comprising the step of administering ribavirin to each patient.

29. The method of claim 17, wherein each patient is administered a solid form of VX-950.

30. The method of claim 29, wherein the solid form is a tablet form.

31. The method of claim 17, wherein each patient is administered VX-950 with food.
